SEQUENCE LISTING (24 sequences)

SEQ ID NO: 1 - DNA, 685 bp, Homo sapiens
ataatagggccggtgctgcctgccgaagccggcggctgagaggcagcgaactcatctttgccagtacaggagcttgtgccgtggcccacagcccacagcccacagccatgggctgggacctgacggtgaagatgctggcgggcaacgaattccaggtgtccctgagcagctccatgtcggtgtcagagctgaaggcgcagatcacccagaagatcggcgtgcacgccttccagcagcgtctggctgtccacccgagcggtgtggcgctgcaggacagggtcccccttgccagccagggcctgggccccggcagcacggtcctgctggtggtggacaaatgcgacgaacctctgagcatcctggtgaggaataacaagggccgcagcagcacctacgaggtacggctgacgcagaccgtggcccacctgaagcagcaagtgagcgggctggagggtgtgcaggacgacctgttctggctgaccttcgaggggaagcccctggaggaccagctcccgctgggggagtacggcctcaagcccctgagcaccgtgttcatgaatctgcgcctgcggggaggcggcacagagcctggcgggcggagctaagggcctccaccagcatccgagcaggatcaagggccggaaataaaggctgttgtaaagagaaaaaaaaaaaaaaaaaaaaa

SEQ ID NO: 2 - protein, 165 aa, Homo sapiens
MGWDLTVKMLAGNEFQVSLSSSMSVSELKAQITQKIGVHAFQQRLAVHPSGVALQDRVPLASQGLGPGSTVLLVVDKCDEPLSILVRNNKGRSSTYEVRLTQTVAHLKQQVSGLEGVQDDLFWLTFEGKPLEDQLPLGEYGLKPLSTVFMNLRLRGGGTEPGGRS

SEQ ID NO: 3 - DNA, 2464 bp, Homo sapiens
actttcctttcccctttcataaaagcacagacctaacagcaccctgggtggaaacctcttcagcatttgcttggaatcagtaagctaaaaacaaaatcaaccgggaccccagcttttcagaactgcagggaaacagccatcatgagtgaggtcaccaagaattccctggagaaaatccttccacagctgaaatgccatttcacctggaacttattcaaggaagacagtgtctcaagggatctagaagatagagtgtgtaaccagattgaatttttaaacactgagttcaaagctacaatgtacaacttgttggcctacataaaacacctagatggtaacaacgaggcagccctggaatgcttacggcaagctgaagagttaatccagcaagaacatgctgaccaagcagaaatcagaagtctagtcacttggggaaactacgcctgggtctactatcacttgggcagactctcagatgctcagatttatgtagataaggtgaaacaaacctgcaagaaattttcaaatccatacagtattgagtattctgaacttgactgtgaggaagggtggacacaactgaagtgtggaagaaatgaaagggcgaaggtgtgttttgagaaggctctggaagaaaagcccaacaacccagaattctcctctggactggcaattgcgatgtaccatctggataatcacccagagaaacagttctctactgatgttttgaagcaggccattgagctgagtcctgataaccaatacgtcaaggttctcttgggcctgaaactgcagaagatgaataaagaagctgaaggagagcagtttgttgaagaagccttggaaaagtctccttgccaaacagatgtcctccgcagtgcagccaaattttacagaagaaaaggtgacctagacaaagctattgaactgtttcaacgggtgttggaatccacaccaaacaatggctacctctatcaccagattgggtgctgctacaaggcaaaagtaagacaaatgcagaatacaggagaatctgaagctagtggaaataaagagatgattgaagcactaaagcaatatgctatggactattcgaataaagctcttgagaagggactgaatcctctgaatgcatactccgatctcgctgagttcctggagacggaatgttatcagacaccattcaataaggaagtccctgatgctgaaaagcaacaatcccatcagcgctactgcaaccttcagaaatataatgggaagtctgaagacactgctgtgcaacatggtttagagggtttgtccataagcaaaaaatcaactgacaaggaagagatcaaagaccaaccacagaatgtatctgaaaatctgcttccacaaaatgcaccaaattattggtatcttcaaggattaattcataagcagaatggagatctgctgcaagcagccaaatgttatgagaaggaactgggccgcctgctaagggatgccccttcaggcataggcagtattttcctgtcagcatctgagcttgaggatggtagtgaggaaatgggccagggcgcagtcagctccagtcccagagagctcctctctaactcagagcaactgaactgagacagaggaggaaaacagagcatcagaagcctgcagtggtggttgtgacgggtaggacgataggaagacagggggccccaacctgggattgctgagcagggaagctttgcatgttgctctaaggtacatttttaaagagttgttttttggccgggcgcagtggctcatgcctgtaatcccagcactttgggaggccgaggtgggcggatcacgaggtctggagtttgagaccatcctggctaacacagtgaaatcccgtctctactaaaaatacaaaaaattagccaggcgtggtggctggcacctgtagtcccagctacttgggaggctgaggcaggagaatggcgtgaacctggaaggaagaggttgcagtgagccaagattgcgcccctgcactccagcctgggcaacagagcaagactccatctcaaaaaaaaaaaaaaaaaaaaaaaagagttgttttctcatgttcattatagttcattacagttacatagtccgaaggtcttacaactaatcactggtagcaataaatgcttcaggcccacatgatgctgattagttctcagttttcattcagttcacaatataaccaccattcctgccctccctgccaagggtcataaatggtgactgcctaacaacaaaatttgcagtctcatctcattttcatccagacttctggaactcaaagattaacttttgactaaccctggaatatctcttatctcacttatagcttcaggcatgtatttatatgtattcttgatagcaataccataatcaatgtgtattcctgatagtaatgctacaataaatccaaacatttcaactctgttaaaaaaaaaaa

SEQ ID NO: 4 - DNA, 2552 bp, Homo sapiens
attttcctcctcccaacgattttaaattagtttcactttccagtttcctcttccttcccctaaaagcaattactcaaaaacggagaaaacatcagctgatgcgtgccctactctcccacccctttatatagttccttcagtatttacttgaggcagacaggaagacttctgaagaacaaatcagcctggtcaccagcttttcggaacagcagagacacagagggcagtcatgagtgaggtcaccaagaattccctggagaaaatccttccacagctgaaatgccatttcacctggaacttattcaaggaagacagtgtctcaagggatctagaagatagagtgtgtaaccagattgaatttttaaacactgagttcaaagctacaatgtacaacttgttggcctacataaaacacctagatggtaacaacgaggcagccctggaatgcttacggcaagctgaagagttaatccagcaagaacatgctgaccaagcagaaatcagaagtctagtcacttggggaaactacgcctgggtctactatcacttgggcagactctcagatgctcagatttatgtagataaggtgaaacaaacctgcaagaaattttcaaatccatacagtattgagtattctgaacttgactgtgaggaagggtggacacaactgaagtgtggaagaaatgaaagggcgaaggtgtgttttgagaaggctctggaagaaaagcccaacaacccagaattctcctctggactggcaattgcgatgtaccatctggataatcacccagagaaacagttctctactgatgttttgaagcaggccattgagctgagtcctgataaccaatacgtcaaggttctcttgggcctgaaactgcagaagatgaataaagaagctgaaggagagcagtttgttgaagaagccttggaaaagtctccttgccaaacagatgtcctccgcagtgcagccaaattttacagaagaaaaggtgacctagacaaagctattgaactgtttcaacgggtgttggaatccacaccaaacaatggctacctctatcaccagattgggtgctgctacaaggcaaaagtaagacaaatgcagaatacaggagaatctgaagctagtggaaataaagagatgattgaagcactaaagcaatatgctatggactattcgaataaagctcttgagaagggactgaatcctctgaatgcatactccgatctcgctgagttcctggagacggaatgttatcagacaccattcaataaggaagtccctgatgctgaaaagcaacaatcccatcagcgctactgcaaccttcagaaatataatgggaagtctgaagacactgctgtgcaacatggtttagagggtttgtccataagcaaaaaatcaactgacaaggaagagatcaaagaccaaccacagaatgtatctgaaaatctgcttccacaaaatgcaccaaattattggtatcttcaaggattaattcataagcagaatggagatctgctgcaagcagccaaatgttatgagaaggaactgggccgcctgctaagggatgccccttcaggcataggcagtattttcctgtcagcatctgagcttgaggatggtagtgaggaaatgggccagggcgcagtcagctccagtcccagagagctcctctctaactcagagcaactgaactgagacagaggaggaaaacagagcatcagaagcctgcagtggtggttgtgacgggtaggacgataggaagacagggggccccaacctgggattgctgagcagggaagctttgcatgttgctctaaggtacatttttaaagagttgttttttggccgggcgcagtggctcatgcctgtaatcccagcactttgggaggccgaggtgggcggatcacgaggtctggagtttgagaccatcctggctaacacagtgaaatcccgtctctactaaaaatacaaaaaattagccaggcgtggtggctggcacctgtagtcccagctacttgggaggctgaggcaggagaatggcgtgaacctggaaggaagaggttgcagtgagccaagattgcgcccctgcactccagcctgggcaacagagcaagactccatctcaaaaaaaaaaaaaaaaaaaaaaaagagttgttttctcatgttcattatagttcattacagttacatagtccgaaggtcttacaactaatcactggtagcaataaatgcttcaggcccacatgatgctgattagttctcagttttcattcagttcacaatataaccaccattcctgccctccctgccaagggtcataaatggtgactgcctaacaacaaaatttgcagtctcatctcattttcatccagacttctggaactcaaagattaacttttgactaaccctggaatatctcttatctcacttatagcttcaggcatgtatttatatgtattcttgatagcaataccataatcaatgtgtattcctgatagtaatgctacaataaatccaaacatttcaactctgttaaaaaaaaaaa

SEQ ID NO: 5 - protein, 490 aa, Homo sapiens
MSEVTKNSLEKILPQLKCHFTWNLFKEDSVSRDLEDRVCNQIEFLNTEFKATMYNLLAYIKHLDGNNEAALECLRQAEELIQQEHADQAEIRSLVTWGNYAWVYYHLGRLSDAQIYVDKVKQTCKKFSNPYSIEYSELDCEEGWTQLKCGRNERAKVCFEKALEEKPNNPEFSSGLAIAMYHLDNHPEKQFSTDVLKQAIELSPDNQYVKVLLGLKLQKMNKEAEGEQFVEEALEKSPCQTDVLRSAAKFYRRKGDLDKAIELFQRVLESTPNNGYLYHQIGCCYKAKVRQMQNTGESEASGNKEMIEALKQYAMDYSNKALEKGLNPLNAYSDLAEFLETECYQTPFNKEVPDAEKQQSHQRYCNLQKYNGKSEDTAVQHGLEGLSISKKSTDKEEIKDQPQNVSENLLPQNAPNYWYLQGLIHKQNGDLLQAAKCYEKELGRLLRDAPSGIGSIFLSASELEDGSEEMGQGAVSSSPRELLSNSEQLN

SEQ ID NO: 6 - protein, 490 aa, Homo sapiens; identical to SEQ ID NO: 5

SEQ ID NO: 7 - DNA, 2037 bp, Homo sapiens
gggaagctcgggccggcagggtttccccgcacgctggcgcccagctcccggcgcggaggccgctgtaagtttcgctttccattcagtggaaaacgaaagctgggcggggtgccacgagcgcggggccagaccaaggcgggcccggagcggaacttcggtcccagctcggtccccggctcagtcccgacgtggaactcagcagcggaggctggacgcttgcatggcgcttgagagattccatcgtgcctggctcacataagcgcttcctggaagtgaagtcgtgctgtcctgaacgcgggccaggcagctgcggcctgggggttttggagtgatcacgaatgagcaaggcgtttgggctcctgaggcaaatctgtcagtccatcctggctgagtcctcgcagtccccggcagatcttgaagaaaagaaggaagaagacagcaacatgaagagagagcagcccagagagcgtcccagggcctgggactaccctcatggcctggttggtttacacaacattggacagacctgctgccttaactccttgattcaggtgttcgtaatgaatgtggacttcaccaggatattgaagaggatcacggtgcccaggggagctgacgagcagaggagaagcgtccctttccagatgcttctgctgctggagaagatgcaggacagccggcagaaagcagtgcggcccctggagctggcctactgcctgcagaagtgcaacgtgcccttgtttgtccaacatgatgctgcccaactgtacctcaaactctggaacctgattaaggaccagatcactgatgtgcacttggtggagagactgcaggccctgtatacgatccgggtgaaggactccttgatttgcgttgactgtgccatggagagtagcagaaacagcagcatgctcaccctcccactttctctttttgatgtggactcaaagcccctgaagacactggaggacgccctgcactgcttcttccagcccagggagttatcaagcaaaagcaagtgcttctgtgagaactgtgggaagaagacccgtgggaaacaggtcttgaagctgacccatttgccccagaccctgacaatccacctcatgcgattctccatcaggaattcacagacgagaaagatctgccactccctgtacttcccccagagcttggatttcagccagatccttccaatgaagcgagagtcttgtgatgctgaggagcagtctggagggcagtatgagctttttgctgtgattgcgcacgtgggaatggcagactccggtcattactgtgtctacatccggaatgctgtggatggaaaatggttctgcttcaatgactccaatatttgcttggtgtcctgggaagacatccagtgtacctacggaaatcctaactaccactggcaggaaactgcatatcttctggtttacatgaagatggagtgctaatggaaatgcccaaaaccttcagagattgacacgctgtcattttccatttccgttcctggatctacggagtcttctaagagattttgcaatgaggagaagcattgttttcaaactatataactgagccttatttataattagggatattatcaaaatatgtaaccatgaggcccctcaggtcctgatcagtcagaatggatgctttcaccagcagacccggccatgtggctgctcggtcctgggtgctcgctgctgtgcaagacattagccctttagttatgagcctgtgggaacttcaggggttcccagtggggagagcagtggcagtgggaggcatctgggggccaaaggtcagtggcagggggtatttcagtattatacaactgctgtgaccagacttgtatactggctgaatatcagtgctgtttgtaatttttcactttgagaaccaacattaattccatatgaatcaagtgttttgtaactgctattcatttattcagcaaatatttattgatcatctcttctccataagatagtgtgataaacacagtcatgaataaagttattttccacaaaa

SEQ ID NO: 8 - protein, 372 aa, Homo sapiens
MSKAFGLLRQICQSILAESSQSPADLEEKKEEDSNMKREQPRERPRAWDYPHGLVGLHNIGQTCCLNSLIQVFVMNVDFTRILKRITVPRGADEQRRSVPFQMLLLLEKMQDSRQKAVRPLELAYCLQKCNVPLFVQHDAAQLYLKLWNLIKDQITDVHLVERLQALYTIRVKDSLICVDCAMESSRNSSMLTLPLSLFDVDSKPLKTLEDALHCFFQPRELSSKSKCFCENCGKKTRGKQVLKLTHLPQTLTIHLMRFSIRNSQTRKICHSLYFPQSLDFSQILPMKRESCDAEEQSGGQYELFAVIAHVGMADSGHYCVYIRNAVDGKWFCFNDSNICLVSWEDIQCTYGNPNYHWQETAYLLVYMKMEC

SEQ ID NO: 9 - DNA, 4127 bp, Homo sapiens
agcagacgagggcttgtgcgagagggggccgggcggctgcagggaaggcggagtccaaggggaaaacgaaactgagaaccagctctcccgaagccgcgggtctccggccggcggcggcggcggcggcggcggcggcgcagtttctggagcaaattcagtttgccttcctggatttgtaaattgtaatgacctcaaaactttagcagttcttccatctgactcaggtttgcttctctggcggtcttcagaatcaacatccacacttccgtgattatctgcgtgcattttggacaaagcttccaaccaggatacgggaagaagaaatggctggtgatctttcagcaggtttcttcatggaggaacttaatacataccgtcagaagcagggagtagtacttaaatatcaagaactgcctaattcaggacctccacatgataggaggtttacatttcaagttataatagatggaagagaatttccagaaggtgaaggtagatcaaagaaggaagcaaaaaatgccgcagccaaattagctgttgagatacttaataaggaaaagaaggcagttagtcctttattattgacaacaacgaattcttcagaaggattatccatggggaattacataggccttatcaatagaattgcccagaagaaaagactaactgtaaattatgaacagtgtgcatcgggggtgcatgggccagaaggatttcattataaatgcaaaatgggacagaaagaatatagtattggtacaggttctactaaacaggaagcaaaacaattggccgctaaacttgcatatcttcagatattatcagaagaaacctcagtgaaatctgactacctgtcctctggttcttttgctactacgtgtgagtcccaaagcaactctttagtgaccagcacactcgcttctgaatcatcatctgaaggtgacttctcagcagatacatcagagataaattctaacagtgacagtttaaacagttcttcgttgcttatgaatggtctcagaaataatcaaaggaaggcaaaaagatctttggcacccagatttgaccttcctgacatgaaagaaacaaagtatactgtggacaagaggtttggcatggattttaaagaaatagaattaattggctcaggtggatttggccaagttttcaaagcaaaacacagaattgacggaaagacttacgttattaaacgtgttaaatataataacgagaaggcggagcgtgaagtaaaagcattggcaaaacttgatcatgtaaatattgttcactacaatggctgttgggatggatttgattatgatcctgagaccagtgatgattctcttgagagcagtgattatgatcctgagaacagcaaaaatagttcaaggtcaaagactaagtgccttttcatccaaatggaattctgtgataaagggaccttggaacaatggattgaaaaaagaagaggcgagaaactagacaaagttttggctttggaactctttgaacaaataacaaaaggggtggattatatacattcaaaaaaattaattcatagagatcttaagccaagtaatatattcttagtagatacaaaacaagtaaagattggagactttggacttgtaacatctctgaaaaatgatggaaagcgaacaaggagtaagggaactttgcgatacatgagcccagaacagatttcttcgcaagactatggaaaggaagtggacctctacgctttggggctaattcttgctgaacttcttcatgtatgtgacactgcttttgaaacatcaaagtttttcacagacctacgggatggcatcatctcagatatatttgataaaaaagaaaaaactcttctacagaaattactctcaaagaaacctgaggatcgacctaacacatctgaaatactaaggaccttgactgtgtggaagaaaagcccagagaaaaatgaacgacacacatgttagagcccttctgaaaaagtatcctgcttctgatatgcagttttccttaaattatctaaaatctgctagggaatatcaatagatatttaccttttattttaatgtttcctttaattttttactatttttactaatctttctgcagaaacagaaaggttttcttctttttgcttcaaaaacattcttacattttactttttcctggctcatctctttattctttttttttttttaaagacagagtctcgctctgttgcccaggctggagtgcaatgacacagtcttggctcactgcaacttctgcctcttgggttcaagtgattctcctgcctcagcctcctgagtagctggattacaggcatgtgccacccacccaactaatttttgtgtttttaataaagacagggtttcaccatgttggccaggctggtctcaaactcctgacctcaagtaatccacctgcctcggcctcccaaagtgctgggattacagggatgagccaccgcgcccagcctcatctctttgttctaaagatggaaaaaccacccccaaattttctttttatactattaatgaatcaatcaattcatatctatttattaaatttctaccgcttttaggccaaaaaaatgtaagatcgttctctgcctcacatagcttacaagccagctggagaaatatggtactcattaaaaaaaaaaaaaaaagtgatgtacaaccacttcggaaaacaatttggcattatctagtaaagttgaatccatgtatacccacatagctatcaattctattcctacatacgtgcttacaagaatgtccataaaaccctgtttataatagccaaaagaacagggaacaaccataatgcacatcaaaagaagaatggattaaaaaaattatattcacacacaggagtactatatagtattgaaaacaattgaagtacagctaaatgtaataacgtaacacaatacaactctcagaaacataatgttaagcgaacaaagcaggttttcagaaaatatatgcagaataattccatttatataaagttccagagcatgcaaaactaaatcattttgtataaaaaacccaacaaatgtgatgagacaataatgggaaggaagggaatgagaaatattaaattctggatggtggttatctttgagggaggggaatgatgtgattggggaaatggactttcaaaggtaatggtaacttccttaagctggatggtaggtccactagtgtttgctgcatagttataccttttatcttaaatacattttgtatctattgtaacaaccactttaaagacaaccgtgctgtaaggcagtagctaaaaacagaaaatagtccatcgggaagggtaagatggctttctgctgagcacagggctagaagtgacagcccagtgggccttccaactatatgccagggtgttagatgagtagagaggagaccacccaggaagtctggacaaggggtctggcatgagctctggagaagatatatttgaggaacatggggtatgctagtttgttgtcctgaattgctgtagagaagataatttaaattgcatcttagaagacgaccctgagggtgaatttcaacttagggcaattgttttagtttgtttcttattggtttaaatggatacttgaagctggataatttataaggaaaagagatttatatgacttacagttctgcaggctgtacaagaaacatggcaccagcatctgcttcttccccggctgcttccactcatggtggaaggtgaaggggagccggatgtgcagagatcatatggcaagagaggaagcaagagagcgagggagaaggtgccaggctctttttaaataaccggctcttgagggaactaatagattgagaactccttgcttctcctccccagcacaccccacccccagggacggcattaatgtattcatgaggggtcttcccccatgacccaaacacctcccatcaggccccacctccaacactgggatcaaatttcaacatgagattttgggggacaaacatgcaaactatagcagcaaccagctaccattctaaaactgccatatgattttaggatttttaaaaagggccaaatttaggttaagcaaaaaaaaaaaaaaaaaa

SEQ ID NO: 10 - DNA, 3697 bp, Homo sapiens
gatacgggaagaagaaatggctggtgatctttcagcaggtttcttcatggaggaacttaatacataccgtcagaagcagggagtagtacttaaatatcaagaactgcctaattcaggacctccacatgataggaggtttacatttcaagttataatagatggaagagaatttccagaaggtgaaggtagatcaaagaaggaagcaaaaaatgccgcagccaaattagctgttgagatacttaataaggaaaagaaggcagttagtcctttattattgacaacaacgaattcttcagaaggattatccatggggaattacataggccttatcaatagaattgcccagaagaaaagactaactgtaaattatgaacagtgtgcatcgggggtgcatgggccagaaggatttcattataaatgcaaaatgggacagaaagaatatagtattggtacaggttctactaaacaggaagcaaaacaattggccgctaaacttgcatatcttcagatattatcagaagaaacctcagtgaaatctgactacctgtcctctggttcttttgctactacgtgtgagtcccaaagcaactctttagtgaccagcacactcgcttctgaatcatcatctgaaggtgacttctcagcagatacatcagagataaattctaacagtgacagtttaaacagttcttcgttgcttatgaatggtctcagaaataatcaaaggaaggcaaaaagatctttggcacccagatttgaccttcctgacatgaaagaaacaaagtatactgtggacaagaggaaggcggagcgtgaagtaaaagcattggcaaaacttgatcatgtaaatattgttcactacaatggctgttgggatggatttgattatgatcctgagaccagtgatgattctcttgagagcagtgattatgatcctgagaacagcaaaaatagttcaaggtcaaagactaagtgccttttcatccaaatggaattctgtgataaagggaccttggaacaatggattgaaaaaagaagaggcgagaaactagacaaagttttggctttggaactctttgaacaaataacaaaaggggtggattatatacattcaaaaaaattaattcatagagatcttaagccaagtaatatattcttagtagatacaaaacaagtaaagattggagactttggacttgtaacatctctgaaaaatgatggaaagcgaacaaggagtaagggaactttgcgatacatgagcccagaacagatttcttcgcaagactatggaaaggaagtggacctctacgctttggggctaattcttgctgaacttcttcatgtatgtgacactgcttttgaaacatcaaagtttttcacagacctacgggatggcatcatctcagatatatttgataaaaaagaaaaaactcttctacagaaattactctcaaagaaacctgaggatcgacctaacacatctgaaatactaaggaccttgactgtgtggaagaaaagcccagagaaaaatgaacgacacacatgttagagcccttctgaaaaagtatcctgcttctgatatgcagttttccttaaattatctaaaatctgctagggaatatcaatagatatttaccttttattttaatgtttcctttaattttttactatttttactaatctttctgcagaaacagaaaggttttcttctttttgcttcaaaaacattcttacattttactttttcctggctcatctctttattctttttttttttttaaagacagagtctcgctctgttgcccaggctggagtgcaatgacacagtcttggctcactgcaacttctgcctcttgggttcaagtgattctcctgcctcagcctcctgagtagctggattacaggcatgtgccacccacccaactaatttttgtgtttttaataaagacagggtttcaccatgttggccaggctggtctcaaactcctgacctcaagtaatccacctgcctcggcctcccaaagtgctgggattacagggatgagccaccgcgcccagcctcatctctttgttctaaagatggaaaaaccacccccaaattttctttttatactattaatgaatcaatcaattcatatctatttattaaatttctaccgcttttaggccaaaaaaatgtaagatcgttctctgcctcacatagcttacaagccagctggagaaatatggtactcattaaaaaaaaaaaaaaaagtgatgtacaaccacttcggaaaacaatttggcattatctagtaaagttgaatccatgtatacccacatagctatcaattctattcctacatacgtgcttacaagaatgtccataaaaccctgtttataatagccaaaagaacagggaacaaccataatgcacatcaaaagaagaatggattaaaaaaattatattcacacacaggagtactatatagtattgaaaacaattgaagtacagctaaatgtaataacgtaacacaatacaactctcagaaacataatgttaagcgaacaaagcaggttttcagaaaatatatgcagaataattccatttatataaagttccagagcatgcaaaactaaatcattttgtataaaaaacccaacaaatgtgatgagacaataatgggaaggaagggaatgagaaatattaaattctggatggtggttatctttgagggaggggaatgatgtgattggggaaatggactttcaaaggtaatggtaacttccttaagctggatggtaggtccactagtgtttgctgcatagttataccttttatcttaaatacattttgtatctattgtaacaaccactttaaagacaaccgtgctgtaaggcagtagctaaaaacagaaaatagtccatcgggaagggtaagatggctttctgctgagcacagggctagaagtgacagcccagtgggccttccaactatatgccagggtgttagatgagtagagaggagaccacccaggaagtctggacaaggggtctggcatgagctctggagaagatatatttgaggaacatggggtatgctagtttgttgtcctgaattgctgtagagaagataatttaaattgcatcttagaagacgaccctgagggtgaatttcaacttagggcaattgttttagtttgtttcttattggtttaaatggatacttgaagctggataatttataaggaaaagagatttatatgacttacagttctgcaggctgtacaagaaacatggcaccagcatctgcttcttccccggctgcttccactcatggtggaaggtgaaggggagccggatgtgcagagatcatatggcaagagaggaagcaagagagcgagggagaaggtgccaggctctttttaaataaccggctcttgagggaactaatagattgagaactccttgcttctcctccccagcacaccccacccccagggacggcattaatgtattcatgaggggtcttcccccatgacccaaacacctcccatcaggccccacctccaacactgggatcaaatttcaacatgagattttgggggacaaacatgcaaactatagcagcaaccagctaccattctaaaactgccatatgattttaggatttttaaaaagggccaaatttaggttaagcaaaaaaaaaaaaaaaaaa

SEQ ID NO: 11 - DNA, 4361 bp, Homo sapiens
agcagacgagggcttgtgcgagagggggccgggcggctgcagggaaggcggagtccaaggggaaaacgaaactgagaaccagctctcccgaagccgcgggtctccggccggcggcggcggcggcggcggcggcggcgcagtttgctcatactttgtgacttgcggtcacagtggcattcagctccacacttggtagaaccacaggcacgacaagcatagaaacatcctaaacaatcttcatcgaggcatcgaggtccatcccaataaaaatcaggagaccctggctatcatagaccttagtcttcgctggtatcactcgtctgtctgaaccagcggttgcatttttttaagccttcttttttctcttttaccagtttctggagcaaattcagtttgccttcctggatttgtaaattgtaatgacctcaaaactttagcagttcttccatctgactcaggtttgcttctctggcggtcttcagaatcaacatccacacttccgtgattatctgcgtgcattttggacaaagcttccaaccaggatacgggaagaagaaatggctggtgatctttcagcaggtttcttcatggaggaacttaatacataccgtcagaagcagggagtagtacttaaatatcaagaactgcctaattcaggacctccacatgataggaggtttacatttcaagttataatagatggaagagaatttccagaaggtgaaggtagatcaaagaaggaagcaaaaaatgccgcagccaaattagctgttgagatacttaataaggaaaagaaggcagttagtcctttattattgacaacaacgaattcttcagaaggattatccatggggaattacataggccttatcaatagaattgcccagaagaaaagactaactgtaaattatgaacagtgtgcatcgggggtgcatgggccagaaggatttcattataaatgcaaaatgggacagaaagaatatagtattggtacaggttctactaaacaggaagcaaaacaattggccgctaaacttgcatatcttcagatattatcagaagaaacctcagtgaaatctgactacctgtcctctggttcttttgctactacgtgtgagtcccaaagcaactctttagtgaccagcacactcgcttctgaatcatcatctgaaggtgacttctcagcagatacatcagagataaattctaacagtgacagtttaaacagttcttcgttgcttatgaatggtctcagaaataatcaaaggaaggcaaaaagatctttggcacccagatttgaccttcctgacatgaaagaaacaaagtatactgtggacaagaggtttggcatggattttaaagaaatagaattaattggctcaggtggatttggccaagttttcaaagcaaaacacagaattgacggaaagacttacgttattaaacgtgttaaatataataacgagaaggcggagcgtgaagtaaaagcattggcaaaacttgatcatgtaaatattgttcactacaatggctgttgggatggatttgattatgatcctgagaccagtgatgattctcttgagagcagtgattatgatcctgagaacagcaaaaatagttcaaggtcaaagactaagtgccttttcatccaaatggaattctgtgataaagggaccttggaacaatggattgaaaaaagaagaggcgagaaactagacaaagttttggctttggaactctttgaacaaataacaaaaggggtggattatatacattcaaaaaaattaattcatagagatcttaagccaagtaatatattcttagtagatacaaaacaagtaaagattggagactttggacttgtaacatctctgaaaaatgatggaaagcgaacaaggagtaagggaactttgcgatacatgagcccagaacagatttcttcgcaagactatggaaaggaagtggacctctacgctttggggctaattcttgctgaacttcttcatgtatgtgacactgcttttgaaacatcaaagtttttcacagacctacgggatggcatcatctcagatatatttgataaaaaagaaaaaactcttctacagaaattactctcaaagaaacctgaggatcgacctaacacatctgaaatactaaggaccttgactgtgtggaagaaaagcccagagaaaaatgaacgacacacatgttagagcccttctgaaaaagtatcctgcttctgatatgcagttttccttaaattatctaaaatctgctagggaatatcaatagatatttaccttttattttaatgtttcctttaattttttactatttttactaatctttctgcagaaacagaaaggttttcttctttttgcttcaaaaacattcttacattttactttttcctggctcatctctttattctttttttttttttaaagacagagtctcgctctgttgcccaggctggagtgcaatgacacagtcttggctcactgcaacttctgcctcttgggttcaagtgattctcctgcctcagcctcctgagtagctggattacaggcatgtgccacccacccaactaatttttgtgtttttaataaagacagggtttcaccatgttggccaggctggtctcaaactcctgacctcaagtaatccacctgcctcggcctcccaaagtgctgggattacagggatgagccaccgcgcccagcctcatctctttgttctaaagatggaaaaaccacccccaaattttctttttatactattaatgaatcaatcaattcatatctatttattaaatttctaccgcttttaggccaaaaaaatgtaagatcgttctctgcctcacatagcttacaagccagctggagaaatatggtactcattaaaaaaaaaaaaaaaagtgatgtacaaccacttcggaaaacaatttggcattatctagtaaagttgaatccatgtatacccacatagctatcaattctattcctacatacgtgcttacaagaatgtccataaaaccctgtttataatagccaaaagaacagggaacaaccataatgcacatcaaaagaagaatggattaaaaaaattatattcacacacaggagtactatatagtattgaaaacaattgaagtacagctaaatgtaataacgtaacacaatacaactctcagaaacataatgttaagcgaacaaagcaggttttcagaaaatatatgcagaataattccatttatataaagttccagagcatgcaaaactaaatcattttgtataaaaaacccaacaaatgtgatgagacaataatgggaaggaagggaatgagaaatattaaattctggatggtggttatctttgagggaggggaatgatgtgattggggaaatggactttcaaaggtaatggtaacttccttaagctggatggtaggtccactagtgtttgctgcatagttataccttttatcttaaatacattttgtatctattgtaacaaccactttaaagacaaccgtgctgtaaggcagtagctaaaaacagaaaatagtccatcgggaagggtaagatggctttctgctgagcacagggctagaagtgacagcccagtgggccttccaactatatgccagggtgttagatgagtagagaggagaccacccaggaagtctggacaaggggtctggcatgagctctggagaagatatatttgaggaacatggggtatgctagtttgttgtcctgaattgctgtagagaagataatttaaattgcatcttagaagacgaccctgagggtgaatttcaacttagggcaattgttttagtttgtttcttattggtttaaatggatacttgaagctggataatttataaggaaaagagatttatatgacttacagttctgcaggctgtacaagaaacatggcaccagcatctgcttcttccccggctgcttccactcatggtggaaggtgaaggggagccggatgtgcagagatcatatggcaagagaggaagcaagagagcgagggagaaggtgccaggctctttttaaataaccggctcttgagggaactaatagattgagaactccttgcttctcctccccagcacaccccacccccagggacggcattaatgtattcatgaggggtcttcccccatgacccaaacacctcccatcaggccccacctccaacactgggatcaaatttcaacatgagattttgggggacaaacatgcaaactatagcagcaaccagctaccattctaaaactgccatatgattttaggatttttaaaaagggccaaatttaggttaagcaaaaaaaaaaaaaaaaaa

SEQ ID NO: 12 - protein, 551 aa, Homo sapiens
MAGDLSAGFFMEELNTYRQKQGVVLKYQELPNSGPPHDRRFTFQVIIDGREFPEGEGRSKKEAKNAAAKLAVEILNKEKKAVSPLLLTTTNSSEGLSMGNYIGLINRIAQKKRLTVNYEQCASGVHGPEGFHYKCKMGQKEYSIGTGSTKQEAKQLAAKLAYLQILSEETSVKSDYLSSGSFATTCESQSNSLVTSTLASESSSEGDFSADTSEINSNSDSLNSSSLLMNGLRNNQRKAKRSLAPRFDLPDMKETKYTVDKRFGMDFKEIELIGSGGFGQVFKAKHRIDGKTYVIKRVKYNNEKAEREVKALAKLDHVNIVHYNGCWDGFDYDPETSDDSLESSDYDPENSKNSSRSKTKCLFIQMEFCDKGTLEQWIEKRRGEKLDKVLALELFEQITKGVDYIHSKKLIHRDLKPSNIFLVDTKQVKIGDFGLVTSLKNDGKRTRSKGTLRYMSPEQISSQDYGKEVDLYALGLILAELLHVCDTAFETSKFFTDLRDGIISDIFDKKEKTLLQKLLSKKPEDRPNTSEILRTLTVWKKSPEKNERHTC

SEQ ID NO: 13 - protein, 510 aa, Homo sapiens
MAGDLSAGFFMEELNTYRQKQGVVLKYQELPNSGPPHDRRFTFQVIIDGREFPEGEGRSKKEAKNAAAKLAVEILNKEKKAVSPLLLTTTNSSEGLSMGNYIGLINRIAQKKRLTVNYEQCASGVHGPEGFHYKCKMGQKEYSIGTGSTKQEAKQLAAKLAYLQILSEETSVKSDYLSSGSFATTCESQSNSLVTSTLASESSSEGDFSADTSEINSNSDSLNSSSLLMNGLRNNQRKAKRSLAPRFDLPDMKETKYTVDKRKAEREVKALAKLDHVNIVHYNGCWDGFDYDPETSDDSLESSDYDPENSKNSSRSKTKCLFIQMEFCDKGTLEQWIEKRRGEKLDKVLALELFEQITKGVDYIHSKKLIHRDLKPSNIFLVDTKQVKIGDFGLVTSLKNDGKRTRSKGTLRYMSPEQISSQDYGKEVDLYALGLILAELLHVCDTAFETSKFFTDLRDGIISDIFDKKEKTLLQKLLSKKPEDRPNTSEILRTLTVWKKSPEKNERHTC

SEQ ID NO: 14 - protein, 551 aa, Homo sapiens; identical to SEQ ID NO: 12

SEQ ID NO: 15 - DNA, 3520 bp, Homo sapiens
tcagtagctgaggctgcggttccccgacgccacgcagctgcgcgcagctggttcccgctctgcagcgcaacgcctgaggcagtgggcgcgctcagtcccgggaccaggcgttctctcctctcgcctctgggcctgggaccccgcaaagcggcgatggagcggaggtcgcggaggaagtcgcggcgcaacgggcgctcgaccgcgggcaaggccgccgcgacccagcccgcgaagtctccgggcgcacagctctggctctttcccagcgccgcgggcctccaccgcgcgctgctccggagggtggaggtgacgcgccaactctgctgctcgccggggcgcctcgcggtcttggaacgcggcggggcgggcgtccaggttcaccagctgctcgccgggagcggcggcgcccggacgccgaaatgcattaaattaggaaaaaacatgaagatacattccgtggaccaaggagcagagcacatgctgattctctcatcagatggaaaaccatttgagtatgacaactatagcatgaaacatctaaggtttgaaagcattttacaagaaaaaaaaataattcagatcacatgtggagattaccattctcttgcactctcaaaaggtggtgagctttttgcctggggacagaacctgcatgggcagcttggagttggaaggaaatttccctcaaccaccacaccacagattgtggagcacctcgcaggagtacccttggctcagatttctgccggagaagcccacagcatggccttatccatgtctggcaacatttattcatggggaaaaaatgaatgtggacaactaggcctgggccacactgagagtaaagatgatccatcccttattgaaggactagacaatcagaaagttgaatttgtcgcttgtggtggctctcacagtgccctactcacacaggatgggctgctgtttactttcggtgctggaaaacatgggcaacttggtcataattcaacacagaatgagctaagaccctgtttggtggctgagcttgttgggtatagagtgactcagatagcatgtggaaggtggcacacacttgcctatgtttctgatttgggaaaggtcttttcctttggttctggaaaagatggacaactgggaaatggtggaacacgtgaccagctgatgccgcttccagtgaaagtatcatcaagtgaagaactcaaacttgaaagccatacctcagaaaaggagttaataatgattgctggagggaatcaaagcattttgctctggataaagaaagagaattcatatgttaatctgaagaggacaattcctactctgaatgaagggactgtaaagagatggattgctgatgtggagactaaacggtggcagagcacaaaaagggaaatccaagagatattttcatctcctgcttgtctaactggaagttttttaaggaaaagaagaactacagaaatgatgcctgtttatttggacttaaataaagcaagaaacatcttcaaggagttaacccaaaaggactggattactaacatgataaccacctgcctcaaagataatctgctcaaaagacttccatttcattctccaccccaagaagctttagaaattttcttccttctcccagaatgtcctatgatgcatatttccaacaactgggagagccttgtggttccatttgcaaaggttgtttgtaaaatgagtgaccagtcttcactggttctggaagagtattgggcaactctgcaagaatccactttcagcaaactggtccagatgtttaaaacagccgtcatatgccagttggattactgggatgaaagtgctgaggagaatggtaatgttcaagctctcctagaaatgttgaagaagctgcacagggtaaaccaggtgaaatgtcaactacctgaaagtattttccaagtagacgaactcttgcaccgtctcaatttttttgtagaagtatgcagaaggtacttgtggaaaatgactgtggacgcttcagaaaatgtacaatgctgcgtcatattcagtcactttccatttatctttaataatctgtcgaaaattaaactactacatacagacacacttttaaaaatagagagtaaaaaacataaagcttatcttaggtcggcagcaattgaggaagaaagagagtctgaattcgctttgaggcccacgtttgatctaacagtcagaaggaatcacttgattgaggatgttttgaatcagctaagtcaatttgagaatgaagacctgaggaaagagttatgggtttcatttagtggagaaattgggtatgacctcggaggagtcaagaaagagttcttctactgtctgtttgcagagatgatccagccggaatatgggatgttcatgtatcctgaaggggcttcctgcatgtggtttcctgtcaagcctaaatttgagaagaaaagatacttcttttttggggttctatgtggactttccctgttcaattgcaatgttgccaaccttcctttcccactggcactgtttaagaaacttttggaccaaatgccatcattggaagacttgaaagaactcagtcctgatttgggaaagaatttgcaaacacttctggatgatgaaggtgataactttgaggaagtattttacatccattttaatgtgcactgggacagaaacgacacaaacttaattcctaatggaagtagcataactgtcaaccagactaacaagagagactatgtttctaagtatatcaattacattttcaacgactctgtaaaggcggtttatgaagaatttcggagaggattttataaaatgtgcgacgaagacattatcaaattattccaccccgaagaactgaaggatgtgattgttggaaatacagattatgattggaaaacatttgaaaagaatgcacgttatgaaccaggatataacagttcacatcccaccatagtgatgttttggaaggctttccacaaattgactctggaagaaaagaaaaaattccttgtatttcttacaggaactgacagactacaaatgaaagatttaaataatatgaaaataacattttgctgtcctgaaagttggaatgaaagagaccctataagagcactgacatgtttcagtgtcctcttcctccctaaatattctacaatggaaacagttgaagaagcgcttcaagaagccatcaacaacaacagaggatttggctgaccagcttgcttgtccaacagccttattttgttgttgttatcgttgttgttgttgttgttgttgttgtttctctactttgttttgttttaggcttttagcagcctgaagccatggtttttcatttctgtctctagtgataagcaggaaagagggatgaagaagagggtttactggccggttagaacccgtgactgtattctctcccttggatacccctatgcctacatcatattccttacctcttttgggaaatatttttcaaaaataaaataaccgaaaaattaacataaaa

SEQ ID NO: 16 - protein, 1024 aa, Homo sapiens
MERRSRRKSRRNGRSTAGKAAATQPAKSPGAQLWLFPSAAGLHRALLRRVEVTRQLCCSPGRLAVLERGGAGVQVHQLLAGSGGARTPKCIKLGKNMKIHSVDQGAEHMLILSSDGKPFEYDNYSMKHLRFESILQEKKIIQITCGDYHSLALSKGGELFAWGQNLHGQLGVGRKFPSTTTPQIVEHLAGVPLAQISAGEAHSMALSMSGNIYSWGKNECGQLGLGHTESKDDPSLIEGLDNQKVEFVACGGSHSALLTQDGLLFTFGAGKHGQLGHNSTQNELRPCLVAELVGYRVTQIACGRWHTLAYVSDLGKVFSFGSGKDGQLGNGGTRDQLMPLPVKVSSSEELKLESHTSEKELIMIAGGNQSILLWIKKENSYVNLKRTIPTLNEGTVKRWIADVETKRWQSTKREIQEIFSSPACLTGSFLRKRRTTEMMPVYLDLNKARNIFKELTQKDWITNMITTCLKDNLLKRLPFHSPPQEALEIFFLLPECPMMHISNNWESLVVPFAKVVCKMSDQSSLVLEEYWATLQESTFSKLVQMFKTAVICQLDYWDESAEENGNVQALLEMLKKLHRVNQVKCQLPESIFQVDELLHRLNFFVEVCRRYLWKMTVDASENVQCCVIFSHFPFIFNNLSKIKLLHTDTLLKIESKKHKAYLRSAAIEEERESEFALRPTFDLTVRRNHLIEDVLNQLSQFENEDLRKELWVSFSGEIGYDLGGVKKEFFYCLFAEMIQPEYGMFMYPEGASCMWFPVKPKFEKKRYFFFGVLCGLSLFNCNVANLPFPLALFKKLLDQMPSLEDLKELSPDLGKNLQTLLDDEGDNFEEVFYIHFNVHWDRNDTNLIPNGSSITVNQTNKRDYVSKYINYIFNDSVKAVYEEFRRGFYKMCDEDIIKLFHPEELKDVIVGNTDYDWKTFEKNARYEPGYNSSHPTIVMFWKAFHKLTLEEKKKFLVFLTGTDRLQMKDLNNMKITFCCPESWNERDPIRALTCFSVLFLPKYSTMETVEEALQEAINNNRGFG

SEQ ID NO: 17 - DNA, 3512 bp, Homo sapiens
aactcagctgagtgttagtcaaagaaggtgtgtcctgctccccaatgacaggttgctcagagactgctgatttccatccctatataaagagagtccctggcatacagagactgctctgctccaggcatctgccacaatgtgggtgcttacacctgctgcttttgctgggaagctcttgagtgtgttcaggcaacctctgagctctctgtggaggagcctggtcccgctgttctgctggctgagggcaaccttctggctgctagctaccaagaggagaaagcagcagctggtcctgagagggccagatgagaccaaagaggaggaagaggaccctcctctgcccaccaccccaaccagcgtcaactatcacttcactcgccagtgcaactacaaatgcggcttctgtttccacacagccaaaacatcctttgtgctgccccttgaggaagcaaagagaggattgcttttgcttaaggaagctggtatggagaagatcaacttttcaggtggagagccatttcttcaagaccggggagaatacctgggcaagttggtgaggttctgcaaagtagagttgcggctgcccagcgtgagcatcgtgagcaatggaagcctgatccgggagaggtggttccagaattatggtgagtatttggacattctcgctatctcctgtgacagctttgacgaggaagtcaatgtccttattggccgtggccaaggaaagaagaaccatgtggaaaaccttcaaaagctgaggaggtggtgtagggattatagagtcgctttcaagataaattctgtcattaatcgtttcaacgtggaagaggacatgacggaacagatcaaagcactaaaccctgtccgctggaaagtgttccagtgcctcttaattgagggtgagaattgtggagaagatgctctaagagaagcagaaagatttgttattggtgatgaagaatttgaaagattcttggagcgccacaaagaagtgtcctgcttggtgcctgaatctaaccagaagatgaaagactcctaccttattctggatgaatatatgcgctttctgaactgtagaaagggacggaaggacccttccaagtccatcctggatgttggtgtagaagaagctataaaattcagtggatttgatgaaaagatgtttctgaagcgaggaggaaaatacatatggagtaaggctgatctgaagctggattggtagagcggaaagtggaacgagacttcaacacaccagtgggaaaactcctagagtaactgccattgtctgcaatactatcccgttggtatttcccagtggctgaaaacctgattttctgctgcacgtggcatctgattacctgtggtcactgaacacacgaataacttggatagcaaatcctgagacaatggaaaaccattaactttacttcattggcttataaccttgttgttattgaaacagcacttctgtttttgagtttgttttagctaaaaagaaggaatacacacaggaataatgaccccaaaaatgcttagataaggcccctatacacaggacctgacatttagctcaatgatgcgtttgtaagaaataagctctagtgatatctgtgggggcaaaatttaatttggatttgattttttaaaacaatgtttactgcgatttctatatttccattttgaaactatttcttgttccaggtttgttcatttgacagagtcagtattttttgccaaatatccagataaccagttttcacatctgagacattacaaagtatctgcctcaattatttctgctggttataatgcttttttttttttgcctttatgccattgcagtcttgtactttttactgtgatgtacagaaatagtcaacagatgtttccaagaacatatgatatgataatcctaccaattttcaagaagtctctagaaagagataacacatggaaagacggtgtggtgcagcccagcccacggtggctgttccatgaatgctggctacctatgtgtgtggtacctgttgtgtccctttctcttcaaagatcctgagcaaaacaaagatacgctttccatttgatgatggagttgacatggaggcagtgcttgcattgctttgttcgcctatcatctggccacatgaggctgtcaagcaaaagaataggagtgtagttgagtagctggttggccctacatctctgagaagtgacggcacactgggttggcataagatatcctaaaatcacgctggaaccttgggcaaggaagaatgtgagcaagagtagagagagtgcctggatttcatgtcagtgaagccaagtcaccatatcatatttttgaatgaactctgagtcagttgaaatagggtaccatctaggtcagtttaagaagagtcagctcagagaaagcaagcataagggaaaatgtcacgtaaactagatcagggaacaaaatcctctccttgtggaaatatcccatgcagtttgttgatacaacttagtatcttattgcctaaaaaaaaatttcttatcattgtttcaaaaaagcaaaatcatggaaaatttttgttgtccaggcaaataaaaggtcattttaatttagctgcaatttcagtgttcctcactaggtggcatttaaatgtcgcctgatgtcattaagcaccatccaaaaagtctgcttcataatctattttcaagacttggtgattctgaaagttttggtttttgtgactttgtttctcaggaaaaaaaatattcctacttaaattttaagtctataattcaatttaaatatgtgtgtgtctcatccaggataggataggttgtcttctattttccattttacctatttactttttttgtaagaaaagagaaaaatgaattctaaagatgttccccatgggttttgattgtgtctaagctatgatgaccttcatataatcagcataaacataaaacaaattttttacttaacatgagtgcactttactaatcctcatggcacagtggctcacgcctgtaatcccagcacttgggaggacaatgtgggtggatcacgaggtcaggagttcgagaacagcctggccaacatggtgaaaccccgtctccactaaaaatacaaaaattagccaggcatggtggcgtacacttgtaattccagctactcaagaggctgaggcaggaggattgcttgaaccctgaaggcagaggttacagagccaagatagcgccactgcactccagcctggatgacagagcaagactccgtctcaaaaaaaaaaaaaaaaaaaagcaagagagttcaactaagaaaggtcacatatgtgaaagcccaaggacactgtttgatatacagcaggtattcaatcagtgttatttgaaaccaaatctgaatttgaagtttgaatcttctgagttggaatgaatttttttctagctgagggaaactgtatttttctttccccaaagaggaatgtaatgtaaagtgaaataaaactataagctatgtt

SEQ ID NO: 18 - protein, 361 aa, Homo sapiens
MWVLTPAAFAGKLLSVFRQPLSSLWRSLVPLFCWLRATFWLLATKRRKQQLVLRGPDETKEEEEDPPLPTTPTSVNYHFTRQCNYKCGFCFHTAKTSFVLPLEEAKRGLLLLKEAGMEKINFSGGEPFLQDRGEYLGKLVRFCKVELRLPSVSIVSNGSLIRERWFQNYGEYLDILAISCDSFDEEVNVLIGRGQGKKNHVENLQKLRRWCRDYRVAFKINSVINRFNVEEDMTEQIKALNPVRWKVFQCLLIEGENCGEDALREAERFVIGDEEFERFLERHKEVSCLVPESNQKMKDSYLILDEYMRFLNCRKGRKDPSKSILDVGVEEAIKFSGFDEKMFLKRGGKYIWSKADLKLDW

SEQ ID NO: 19 - DNA, 2983 bp, Homo sapiens
gttcgggcccgagaacctgcgtctcccgcgagttcccgcgaggcaagtgctgcaggtgcggggccaggagctaggtttcgtttctgcgcccggagccgccctcagcacagggtctgtgagtttcatttcttcgcggcgcggggcggggctgggcgcggggtgaaagaggcgaagcgagagcggaggccgcactccagcactgcgcagggaccgccttggaccgcagttgccggccaggaatcccagtgtcacggtggacacgcctccctcgcgcccttgccgcccacctgctcacccagctcaggggctttggaattctgtggccacactgcgaggagatcggttctgggtcggaggctacaggaagactcccactccctgaaatctggagtgaagaacgccgccatccagccaccattccaaggaggtgcaggagaacagctctgtgataccatttaacttgttgacattacttttatttgaaggaacgtatattagagcttactttgcaaagaaggaagatggttgtttccgaagtggacatcgcaaaagctgatccagctgctgcatcccaccctctattactgaatggagatgctactgtggcccagaaaaatccaggctcggtggctgagaacaacctgtgcagccagtatgaggagaaggtgcgcccctgcatcgacctcattgactccctgcgggctctaggtgtggagcaggacctggccctgccagccatcgccgtcatcggggaccagagctcgggcaagagctccgtgttggaggcactgtcaggagttgcccttcccagaggcagcgggatcgtgaccagatgcccgctggtgctgaaactgaagaaacttgtgaacgaagataagtggagaggcaaggtcagttaccaggactacgagattgagatttcggatgcttcagaggtagaaaaggaaattaataaagcccagaatgccatcgccggggaaggaatgggaatcagtcatgagctaatcaccctggagatcagctcccgagatgtcccggatctgactctaatagaccttcctggcataaccagagtggctgtgggcaatcagcctgctgacattgggtataagatcaagacactcatcaagaagtacatccagaggcaggagacaatcagcctggtggtggtccccagtaatgtggacatcgccaccacagaggctctcagcatggcccaggaggtggaccccgagggagacaggaccatcggaatcttgacgaagcctgatctggtggacaaaggaactgaagacaaggttgtggacgtggtgcggaacctcgtgttccacctgaagaagggttacatgattgtcaagtgccggggccagcaggagatccaggaccagctgagcctgtccgaagccctgcagagagagaagatcttctttgagaaccacccatatttcagggatctgctggaggaaggaaaggccacggttccctgcctggcagaaaaacttaccagcgagctcatcacacatatctgtaaatctctgcccctgttagaaaatcaaatcaaggagactcaccagagaataacagaggagctacaaaagtatggtgtcgacataccggaagacgaaaatgaaaaaatgttcttcctgatagataaagttaatgcctttaatcaggacatcactgctctcatgcaaggagaggaaactgtaggggaggaagacattcggctgtttaccagactccgacacgagttccacaaatggagtacaataattgaaaacaattttcaagaaggccataaaattttgagtagaaaaatccagaaatttgaaaatcagtatcgtggtagagagctgccaggctttgtgaattacaggacatttgagacaatcgtgaaacagcaaatcaaggcactggaagagccggctgtggatatgctacacaccgtgacggatatggtccggcttgctttcacagatgtttcgataaaaaattttgaagagttttttaacctccacagaaccgccaagtccaaaattgaagacattagagcagaacaagagagagaaggtgagaagctgatccgcctccacttccagatggaacagattgtctactgccaggaccaggtatacaggggtgcattgcagaaggtcagagagaaggagctggaagaagaaaagaagaagaaatcctgggattttggggctttccagtccagctcggcaacagactcttccatggaggagatctttcagcacctgatggcctatcaccaggaggccagcaagcgcatctccagccacatccctttgatcatccagttcttcatgctccagacgtacggccagcagcttcagaaggccatgctgcagctcctgcaggacaaggacacctacagctggctcctgaaggagcggagcgacaccagcgacaagcggaagttcctgaaggagcggcttgcacggctgacgcaggctcggcgccggcttgcccagttccccggttaaccacactctgtccagccccgtagacgtgcacgcacactgtctgcccccgttcccgggtagccactggactgacgacttgagtgctcagtagtcagactggatagtccgtctctgcttatccgttagccgtggtgatttagcaggaagctgtgagagcagtttggtttctagcatgaagacagagccccaccctcagatgcacatgagctggcgggattgaaggatgctgtcttcgtactgggaaagggattttcagccctcagaatcgctccaccttgcagctctccccttctctgtattcctagaaactgacacatgctgaacatcacagcttatttcctcatttttataatgtcccttcacaaacccagtgttttaggagcatgagtgccgtgtgtgtgcgtcctgtcggagccctgtctcctctctctgtaataaactcatttctagcagacaaaaaaaaaaaaaaaaaa

SEQ ID NO: 20 - protein, 662 aa, Homo sapiens
MVVSEVDIAKADPAAASHPLLLNGDATVAQKNPGSVAENNLCSQYEEKVRPCIDLIDSLRALGVEQDLALPAIAVIGDQSSGKSSVLEALSGVALPRGSGIVTRCPLVLKLKKLVNEDKWRGKVSYQDYEIEISDASEVEKEINKAQNAIAGEGMGISHELITLEISSRDVPDLTLIDLPGITRVAVGNQPADIGYKIKTLIKKYIQRQETISLVVVPSNVDIATTEALSMAQEVDPEGDRTIGILTKPDLVDKGTEDKVVDVVRNLVFHLKKGYMIVKCRGQQEIQDQLSLSEALQREKIFFENHPYFRDLLEEGKATVPCLAEKLTSELITHICKSLPLLENQIKETHQRITEELQKYGVDIPEDENEKMFFLIDKVNAFNQDITALMQGEETVGEEDIRLFTRLRHEFHKWSTIIENNFQEGHKILSRKIQKFENQYRGRELPGFVNYRTFETIVKQQIKALEEPAVDMLHTVTDMVRLAFTDVSIKNFEEFFNLHRTAKSKIEDIRAEQEREGEKLIRLHFQMEQIVYCQDQVYRGALQKVREKELEEEKKKKSWDFGAFQSSSATDSSMEEIFQHLMAYHQEASKRISSHIPLIIQFFMLQTYGQQLQKAMLQLLQDKDTYSWLLKERSDTSDKRKFLKERLARLTQARRRLAQFPG

SEQ ID NO: 21 - DNA, 2683 bp, Homo sapiens
tttcttcgcggcgcggggcggggctgggcgcggggtgaaagaggcgaagcgagagcggaggccgcactccagcactgcgcagggaccggaattctgtggccacactgcgaggagatcggttctgggtcggaggctacaggaagactcccactccctgaaatctggagtgaagaacgccgccatccagccaccattccaagcttactttgcaaagaaggaagatggttgtttccgaagtggacatcgcaaaagctgatccagctgctgcatcccaccctctattactgaatggagatgctactgtggcccagaaaaatccaggctcggtggctgagaacaacctgtgcagccagtatgaggagaaggtgcgcccctgcatcgacctcattgactccctgcgggctctaggtgtggagcaggacctggccctgccagccatcgccgtcatcggggaccagagctcgggcaagagctccgtgttggaggcactgtcaggagttgcccttcccagaggcagcgggatcgtgaccagatgcccgctggtgctgaaactgaagaaacttgtgaacgaagataagtggagaggcaaggtcagttaccaggactacgagattgagatttcggatgcttcagaggtagaaaaggaaattaataaagcccagaatgccatcgccggggaaggaatgggaatcagtcatgagctaatcaccctggagatcagctcccgagatgtcccggatctgactctaatagaccttcctggcataaccagagtggctgtgggcaatcagcctgctgacattgggtataagatcaagacactcatcaagaagtacatccagaggcaggagacaatcagcctggtggtggtccccagtaatgtggacatcgccaccacagaggctctcagcatggcccaggaggtggaccccgagggagacaggaccatcggaatcttgacgaagcctgatctggtggacaaaggaactgaagacaaggttgtggacgtggtgcggaacctcgtgttccacctgaagaagggttacatgattgtcaagtgccggggccagcaggagatccaggaccagctgagcctgtccgaagccctgcagagagagaagatcttctttgagaaccacccatatttcagggatctgctggaggaaggaaaggccacggttccctgcctggcagaaaaacttaccagcgagctcatcacacatatctgtaaatctctgcccctgttagaaaatcaaatcaaggagactcaccagagaataacagaggagctacaaaagtatggtgtcgacataccggaagacgaaaatgaaaaaatgttcttcctgatagataaagttaatgcctttaatcaggacatcactgctctcatgcaaggagaggaaactgtaggggaggaagacattcggctgtttaccagactccgacacgagttccacaaatggagtacaataattgaaaacaattttcaagaaggccataaaattttgagtagaaaaatccagaaatttgaaaatcagtatcgtggtagagagctgccaggctttgtgaattacaggacatttgagacaatcgtgaaacagcaaatcaaggcactggaagagccggctgtggatatgctacacaccgtgacggatatggtccggcttgctttcacagatgtttcgataaaaaattttgaagagttttttaacctccacagaaccgccaagtccaaaattgaagacattagagcagaacaagagagagaaggtgagaagctgatccgcctccacttccagatggaacagattgtctactgccaggaccaggtatacaggggtgcattgcagaaggtcagagagaaggagctggaagaagaaaagaagaagaaatcctgggattttggggctttccagtccagctcggcaacagactcttccatggaggagatctttcagcacctgatggcctatcaccaggaggccagcaagcgcatctccagccacatccctttgatcatccagttcttcatgctccagacgtacggccagcagcttcagaaggccatgctgcagctcctgcaggacaaggacacctacagctggctcctgaaggagcggagcgacaccagcgacaagcggaagttcctgaaggagcggcttgcacggctgacgcaggctcggcgccggcttgcccagttccccggttaaccacactctgtccagccccgtagacgtgcacgcacactgtctgcccccgttcccgggtagccactggactgacgacttgagtgctcagtagtcagactggatagtccgtctctgcttatccgttagccgtggtgatttagcaggaagctgtgagagcagtttggtttctagcatgaagacagagccccaccctcagatgcacatgagctggcgggattgaaggatgctgtcttcgtactgggaaagggattttcagccctcagaatcgctccaccttgcagctctccccttctctgtattcctagaaactgacacatgctgaacatcacagcttatttcctcatttttataatgtcccttcacaaacccagtgttttaggagcatgagtgccgtgtgtgtgcgtcctgtcggagccctgtctcctctctctgtaataaactcatttctagcagacaaaaaaaaaaaaaaaaaa

SEQ ID NO: 22 - protein, 662 aa, Homo sapiens; identical to SEQ ID NO: 20

SEQ ID NO: 23 - DNA, 3409 bp, Homo sapiens
aaattcgcggtgggggcggagagcgcagggagaagtaagcccagtgcaggatcctgaggcccgtgtttgcaggaccagggccggccttccgattccccattcattccagaagcaccgaaccacgctgtgcccggatcccaagtgcagcggcacccagcgtgggcctggggttgccggttgacccggtcctcagcctggtagcagaggccaggccagtgccacaaggcacctaagtccacctgggcctggagcaggacaggttgcaaaagaaaatatctcgggacccccaaactccttatgctaagggaaacatcgagcctgggaactgagccatcaacgctgccattctttttcccaaacagaaccctgttgtcagaggtacacccagagcaactccacaccgggtgcatgccacagcaactccatcttaaataggagctggtaaaacgaggctgatacctactgggctgcattcccagacggcatagcgaggaggtgctgaagagcgcaggtttggagaatgatcacctggattggaaccatagctctaccaatatggaacccagctccttaggcctcggtcttctcatggagaacatggtgtgataatcctactcctctgggagggtggctgttaagccttggaccgcagttgccggccaggaatcccagtgtcacggtggacacgcctccctcgcgcccttgccgcccacctgctcacccagctcaggggctttggaattctgtggccacactgcgaggagatcggttctgggtcggaggctacaggaagactcccactccctgaaatctggagtgaagaacgccgccatccagccaccattccaaggaggtgcaggagaacagctctgtgataccatttaacttgttgacattacttttatttgaaggaacgtatattagagcttactttgcaaagaaggaagatggttgtttccgaagtggacatcgcaaaagctgatccagctgctgcatcccaccctctattactgaatggagatgctactgtggcccagaaaaatccaggctcggtggctgagaacaacctgtgcagccagtatgaggagaaggtgcgcccctgcatcgacctcattgactccctgcgggctctaggtgtggagcaggacctggccctgccagccatcgccgtcatcggggaccagagctcgggcaagagctccgtgttggaggcactgtcaggagttgcccttcccagaggcagcgggatcgtgaccagatgcccgctggtgctgaaactgaagaaacttgtgaacgaagataagtggagaggcaaggtcagttaccaggactacgagattgagatttcggatgcttcagaggtagaaaaggaaattaataaagcccagaatgccatcgccggggaaggaatgggaatcagtcatgagctaatcaccctggagatcagctcccgagatgtcccggatctgactctaatagaccttcctggcataaccagagtggctgtgggcaatcagcctgctgacattgggtataagatcaagacactcatcaagaagtacatccagaggcaggagacaatcagcctggtggtggtccccagtaatgtggacatcgccaccacagaggctctcagcatggcccaggaggtggaccccgagggagacaggaccatcggaatcttgacgaagcctgatctggtggacaaaggaactgaagacaaggttgtggacgtggtgcggaacctcgtgttccacctgaagaagggttacatgattgtcaagtgccggggccagcaggagatccaggaccagctgagcctgtccgaagccctgcagagagagaagatcttctttgagaaccacccatatttcagggatctgctggaggaaggaaaggccacggttccctgcctggcagaaaaacttaccagcgagctcatcacacatatctgtaaatctctgcccctgttagaaaatcaaatcaaggagactcaccagagaataacagaggagctacaaaagtatggtgtcgacataccggaagacgaaaatgaaaaaatgttcttcctgatagataaagttaatgcctttaatcaggacatcactgctctcatgcaaggagaggaaactgtaggggaggaagacattcggctgtttaccagactccgacacgagttccacaaatggagtacaataattgaaaacaattttcaagaaggccataaaattttgagtagaaaaatccagaaatttgaaaatcagtatcgtggtagagagctgccaggctttgtgaattacaggacatttgagacaatcgtgaaacagcaaatcaaggcactggaagagccggctgtggatatgctacacaccgtgacggatatggtccggcttgctttcacagatgtttcgataaaaaattttgaagagttttttaacctccacagaaccgccaagtccaaaattgaagacattagagcagaacaagagagagaaggtgagaagctgatccgcctccacttccagatggaacagattgtctactgccaggaccaggtatacaggggtgcattgcagaaggtcagagagaaggagctggaagaagaaaagaagaagaaatcctgggattttggggctttccagtccagctcggcaacagactcttccatggaggagatctttcagcacctgatggcctatcaccaggaggccagcaagcgcatctccagccacatccctttgatcatccagttcttcatgctccagacgtacggccagcagcttcagaaggccatgctgcagctcctgcaggacaaggacacctacagctggctcctgaaggagcggagcgacaccagcgacaagcggaagttcctgaaggagcggcttgcacggctgacgcaggctcggcgccggcttgcccagttccccggttaaccacactctgtccagccccgtagacgtgcacgcacactgtctgcccccgttcccgggtagccactggactgacgacttgagtgctcagtagtcagactggatagtccgtctctgcttatccgttagccgtggtgatttagcaggaagctgtgagagcagtttggtttctagcatgaagacagagccccaccctcagatgcacatgagctggcgggattgaaggatgctgtcttcgtactgggaaagggattttcagccctcagaatcgctccaccttgcagctctccccttctctgtattcctagaaactgacacatgctgaacatcacagcttatttcctcatttttataatgtcccttcacaaacccagtgttttaggagcatgagtgccgtgtgtgtgcgtcctgtcggagccctgtctcctctctctgtaataaactcatttctagcagacaaaaaaaaaaaaaaaaaa

SEQ ID NO: 24 - protein, 662 aa, Homo sapiens; identical to SEQ ID NO: 20

(19) United States
(12) Patent Application Publication
(10) Pub. No.: US 2018/0305761 A1
(43) Pub. Date: Oct. 25, 2018

(54) METHODS AND KITS FOR PREDICTION AND DIAGNOSIS OF HUMAN CYTOMEGALOVIRUS (hCMV) CONGENITAL TRANSMISSION

(71) Applicant: GENEFRON LTD., Har Adar (IL)

(72) Inventor: Yoav SMITH, Jerusalem (IL)

(21) Appl. No.: 15/766,952

(22) PCT Filed: Oct. 9, 2016
(86) PCT No.: PCT/IL2016/051097

§ 371 Date: Apr. 9, 2018

Related U.S. Application Data
(60) Provisional application No. 62/353,624, filed on Jun. 23, 2016.
(60) Provisional application No. 62/239,609, filed on Oct. 9, 2015.

(51) Int. Cl. C12Q 1/6883; C12Q 1/6853; C12Q 1/6851; G06F 19/20

(52) U.S. Cl. CPC C12Q 1/6883; C12Q 1/6853; C12Q 2565/518; G06F 19/20; C12Q 1/6851

(57) ABSTRACT

The present invention relates to methods and kits for the prediction and diagnosis of intrauterine-transmission of viral pathogens, specifically, hCMV in a mammalian subject, by calculating the ability of a subject to prevent transmission of said hCMV based on determining the expression of ISG15, IFIT3 and USP18 genes and optionally of EIF2AK2, HERC5, RSAD2 and MX1 genes in a sample of said subject.

### FIELD OF THE INVENTION

The invention relates to diagnosis of viral pathogens, specifically, human cytomegalovirus (hCMV) transmission. More specifically, the invention provides methods and kits for prediction and detection of intrauterine-transmission of hCMV during early gestation in hCMV infected mammalian subjects.

### RELATED APPLICATIONS

This application is a National Phase of PCT Patent Application No. PCT/IL2016/051097 having International filing date of Oct. 9, 2016, which claims the benefit of priority of U.S. Patent Application Nos. 62/353,624 filed on Jun. 23, 2016 and 62/239,609 filed on Oct. 9, 2015. The contents of the above applications are all incorporated by reference as if fully set forth herein in their entirety.

### BACKGROUND ART

References considered to be relevant as background to the presently disclosed subject matter are listed below:
1. Dollard S C, Grosse S D, Ross D S (2007) New estimates of the prevalence of neurological and sensory sequalae and mortality associated with congenital cytomegalovirus infection. Rev Med Virol 17:355-363
2. Kenneson A, Cannon M J (2007) Review and meta-analysis of the epidemiology of congenital cytomegalovirus (CMV) infection. Rev Med Virol 17:253-276
3. Kern E R (2006) Pivotal role of animal models in the development of new therapies for cytomegalovirus infections. Antiviral Res 71: 164-171
4. Manicklal S, Emery V C, Lazzarotto T, Boppana S B, Gupta R K (2013) The “silent” global burden of congenital cytomegalovirus. Clin Microbiol Rev 26:86-102
5. Nelson C T, Demmler G J (1997) Cytomegalovirus infection in the pregnant mother, fetus, and newborn infant. Clin Perinatol 24:151-160
6. Stagno S, Cloud G, Pass R F, Britt W J, Alford C A (1984) Factors associated with primary cytomegalovirus infection during pregnan-cy. J Med Virol 13:347-353
7. de Vries JJC, Vossen ACTM, Kroes ACM, van der Zeijst BAM. Implementing neonatal screening for congenital cyto-megalovirus: addressing the deafness of policy makers. Rev Med Virol 2011; 21:54-61.
8. Cannon M J, Davis K F (2005) Washing our hands of the congenital cytomegalovirus disease epidemic. BMC Public Health 5:70
9. Grosse S D, Ross D S, Dollard S C. Congenital cytomegalovirus (CMV) infection as a cause of permanent bilateral hearing loss: a quantitative assessment. J Clin Virol 2008; 41(2): 57-62.
10. Ludwig A, Hengel H. Epidemiological impact and disease burden of congenital cytomegalovirus infection in Europe. Euro Surveill 2009; 14(9): 26-32.
11. Longo S, Borghesi A, Tzialla C, Stronati M (2014) IUGR and infections. Early Hum Dev 90(Suppl 1):542-544
12. Pereira L, Petitt M, Fong A, Tsuge M, Tabata T, Fang-Hoover J et al (2014) Intrauterine growth restriction caused by underlying congen-ital cytomegalovirus infection. J Infect Dis 209:1573-1584.
13. Nigro G, Adler S P, La Torre R, Best A M, Congenital Cytomegalovirus Collaborating Group (2005) Passive immuniza-tion during pregnancy for congenital cytomegalovirus infection. N Engl J Med 353:1350-1362
14. Revello M G, Lazzarotto T, Guerra B, Spinillo A, Ferrazzi E, Kustermann A et al (2014) A randomized trial of hyperimmune globulin to prevent congenital cytomegalovirus. N Engl J Med 370: 1316-1326
15. Stagno S. Cytomegalovirus. In: Remington J S, Klein J O, eds. Infectious diseases of the fetus and newborn infant. 5th ed. Phila-delphia: W.B. Saunders, 2001:389-424.
16. Stagno S, Whitley R J. Herpesvirus infec-tions of pregnancy. I. Cytomegalovirus and Epstein-Barr virus infections. N Engl J Med 1985; 313:1270-4.
17. Stagno S, Pass R F, Cloud G, et al. Primary cytomegalovirus infection in pregnancy: inci-dence, transmission to fetus, and clinical out-come. JAMA 1986; 256:1904-8.
18. C M, X.; Churchill, G. A. (2003). “Statistical tests for differential expression in cDNA microarray experiments”. Genome Biology 4 (4): 210. doi:10.1186/gb-2003-4-4-210. PMC 154570. PMID 12702200.
19. Mindy Miller-Kittrell and Tim E Sparer. Feeling manipulated: cytomegalovirus immune manipulation. Virology Journal 2009, 6:4 doi:10.1186/1743-422X-6-4.
20. Cromer D, Siok-Keen Tey, Rajiv Khanna, Miles P. Davenporta Estimating Cytomegalovirus Growth Rates by Using Only a Single Point. Journal of Virol 3376-3381 2013
21. Ying Fang, Peifang Ye, Xiaobo Wang, Xiao Xu, and William Reisen. Real-time monitoring of flavivirus induced cytopathogenesis using cell electric impedance technology J Virol Methods. 2011 May; 173(2): 251-258

Acknowledgement of the above references herein is not to be inferred as meaning that these are in any way relevant to the patentability of the presently disclosed subject matter.

### BACKGROUND OF THE INVENTION

Cytomegalovirus (CMV) is the most common cause of congenital infection in the developed world, affecting 0.5-2% of all live births in the United States and Europe¹⁻⁴. Fetal CMV infection can cause a variety of long-term disabilities including mental, hearing and visual impairments⁵⁻⁷. Severe disabilities caused by congenital CMV infection threaten more children than several well-known childhood maladies such as Down's syndrome or fetal alcohol syndrome^{4,8}.

Intrauterine CMV transmission occurs mainly during primary maternal infection, with a maternal-fetal transmission rate of about 40%^{8,9}. The mechanisms dictating CMV intrauterine transmission are unknown. However, transmission is thought to be dependent on multiple factors, including maternal and fetal immune systems, placental factors, maternal viral load and viral strain⁹⁻¹³.

A large number of studies have demonstrated the essential role of T-cell immunity in the control of CMV infection¹². It was shown that women with primary CMV infection transmitting the virus to the fetus usually display a delayed T-cell lymphoproliferative response to CMV, as compared with non-transmitting women¹⁴⁻¹⁷. In addition, it has also been reported that circulating CMV-specific effector memory T cells (TEM) may revert to the CD45RA+ phenotype, which is associated with control of CMV viremia and mother-to-fetus transmission¹⁸. Importantly, individual immune response heterogeneity precludes predicting fetal CMV transmission.

In the absence of effective means to prevent the congenital hCMV transmission, while approximately 40 percent (REF) of primary hCMV infected women transmit the virus to their fetus early and accurate prediction of intrauterine-transmission during the early stages of pregnancy is of extreme clinical importance.

### SUMMARY OF THE INVENTION

A first aspect of the invention relates to a method for the prediction and diagnosis of intrauterine-transmission of a viral pathogen, specifically, human cytomegalovirus (hCMV) in a mammalian subject. In more specific embodiments, the method of the invention may comprise the following steps: In a first step (a), determining the level of expression of at least one of ISG15 ubiquitin-like modifier (ISG15), Interferon-induced protein with tetratricopeptide repeats 3 (IFIT3), Ubiquitin specific peptidase 18 (USP18), Eukaryotic Translation Initiation Factor 2 Alpha Kinase 2 (EIF2AK2), HECT And RLD Domain Containing E3 Ubiquitin Protein Ligase 5 (HERC5), Radical S-Adenosyl Methionine Domain Containing 2 (RSAD2) and MX Dynamin Like GTPase 1 (MX1) genes in a biological sample of the subject, to obtain an expression value Exₛₐₘₚ in the sample. The next step (b) involves calculating the M value of the sample (Mₛₐₘₚ). It should be noted that the M value of the sample indicates the ability of the examined subject to prevent intrauterine-transmission of said viral pathogen, specifically, hCMV. In the next step (c), providing a standard M (M_{stand}) value of non-transmitting subjects. It should be noted that the standard M value indicates the minimal ability required for preventing intrauterine-transmission of the viral pathogen, specifically, hCMV. The final step (d) involves determining if the M value of the sample (Mₛₐₘₚ) calculated in step (b) is any one of positive or negative with respect to the standard M (M_{stand}) value of non-transmitting subjects provided in (c). Thus, wherein a positive value of Mₛₐₘₚ indicates that the subject is a non-transmitting subject and a negative value of Mₛₐₘₚ indicates that the subject is a viral pathogen transmitting subject, specifically, hCMV transmitting subject, thereby predicting intrauterine-transmission of the viral pathogen in the subject.

A second aspect of the invention relates to a kit comprising detecting molecules specific for determining the level of expression of ISG15, IFIT3 and USP18, and optionally, EIF2AK2, HERC5, RSAD2 and MX1 genes in a biological sample.

In a further aspect, the invention provides a prognostic composition comprising detecting molecules specific for determining the level of expression of ISG15, IFIT3 and USP18, and optionally, EIF2AK2, HERC5, RSAD2 and MX1 genes in a biological sample.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1G. Analysis of five microarray datasets of hCMV infected subjects

FIG. 1A is a volcano plot of the combined data obtained from (I) GSE14490; analysis of dendritic cells derived from 6 mock infected and matching 6 hCMV infected donors, 6 hpi. (II) GSE14816; analysis of dendritic cells derived from 3 mock infected and matching 3 hCMV infected donors, 24 hpi. (III) GSE17948; analysis of monocytes derived from 4 mock infected and matching 4 hCMV infected donors, 24 hpi. (IV) GSE19772; analysis of monocytes derived from 2 mock infected and matching 2 hCMV infected donors, 48 hpi. (V) GSE14408; analysis of monocytes cells derived from 6 mock infected and matching 6 hCMV infected donors, 4 hpi;

FIG. 1B STRING (Search tool for the retrieval of interacting genes/proteins-Version 9.1) analysis of the top ranging genes obtained from the volcano plot;

FIGS. 1C to 1G ISG15 gene expression before and following hCMV infection from the 5 datasets described in FIG. 1A, respectively.

FIG. 2. Simulation of the mathematical model for hCMV infection (K=2), the data shown is for four different M values of: 0.495, 0.65, 0.80 and 0.95.

FIG. 3A-3C. qRT PCR results of 18 pregnant women infected with hCMV

FIG. 3A shows sum of three selected genes (IFIT3, ISG15 and USP18), after Zscore normalization;

FIG. 3B box plot of the three selected genes as in FIG. 3A summarized in Table 1 allows the prediction of congenital hCMV transmission to fetus according to Table 2. Graph shows the distribution between 5 and 13 transmitter and non-transmitters respectively;

FIG. 3C Receiver operating characteristic (ROC) plot of the normalized sum of the three genes as described in FIG. 3A.

FIG. 4A-4C. Calculation of M using measured RT-PCR levels of ISG15 in hCMV infected pregnant women

FIG. 4A shows the levels of ISG15 gene in 18 hCMV infected pregnant women as measured by RT-PCR;

FIG. 4B shows the M values calculated for each subject;

FIG. 4C illustrates the calculated M values as compared to measured ISG15 expression levels of the 18 hCMV infected pregnant women.

FIGS. 5A-5G. Normalized expression levels of genes from pregnant women infected with hCMV

FIG. 5A shows the normalized expression levels of IFIT3 gene; FIG. 5B shows the normalized expression levels of USP18 gene; FIG. 5C shows the normalized expression levels of ISG15 gene; FIG. 5D shows the normalized expression levels of USP18 gene; FIG. 5E shows the normalized expression levels of HERC5 gene; FIG. 5F shows the normalized expression levels of RSAD2 gene; FIG. 5G shows the normalized expression levels of MX1 gene.

FIGS. 6A and 6B. Normalized expression levels of genes from pregnant women infected with hCMV

FIG. 6A shows sum of seven selected genes (ISG15, IFIT3, USP18 EIF2AK2, HERC5, RSAD2 and MX1);

FIG. 6B shows sum of even selected genes (ISG15, IFIT3, USP18 EIF2AK2, HERC5, RSAD2 and MX1) relative to a selected threshold shown as a dashed box.

### DETAILED DESCRIPTION OF THE INVENTION

Human cytomegalovirus (hCMV) is the most common cause of congenital infection in humans, bearing potential serious consequences to the newborns. As not all sera-positive hCMV infected pregnant-women transmit the virus to fetus, it is extremely important to identify those conferring the highest risk of congenital infection and disease.

In the present invention, the inventor has used computational tools and identified an arsenal of genes that is differently expressed in woman that transmitted hCMV to their fetus and women who did not.

As shown herein in the Examples below, the inventors combined a mathematical model for simulation of interferon (IFN) signaling genes responding to hCMV infection with accumulated data from array-datasets related to hCMV infection. The inventors recognized a set of biomarker genes for predicting congenital transmission of the virus. Retrospective validation of the inventors prediction-model was performed by qRT-PCR using total RNA extracted from PBMC obtained from 30 hCMV sera-positive pregnant women, followed by assessing the clinical conditions of the neonates and the viral load in their urine. Un-bias searching for common genes stimulated in hCMV infected pregnant women by screening microarray datasets, revealed a set of genes that are overexpressed, all of which associated with IFN signaling.

Comparing the value of the variables before and after hCMV infection (delta), enabled to distinguish between two populations, those with high- and low-expression of the biomarkers. In a tight correlation to the mathematical model, qRT-PCR analyses of 30 sera-positive pregnant women with detective virus load (≥200 viral copies/ml blood) clearly demonstrates that mothers who delivered infected newborn had a high expression levels of the bio-markers, in significant difference from non-transferring women, who had low expression levels of these genes.

For assessing risk of intrauterine-transmission of hCMV in a pregnant woman, the qRT-PCR analyses of 1, 2, 3, 4, 5, 6, 7 or more biomarkers, combined with the mathematical test, provided a high confident prediction (over 94% accuracy) of the fetus providence. The results presented enable development of a personal diagnostic kits and methods for prediction the probability of hCMV infected pregnant women to transmit the virus to their fetus.

Following the searching of highly expressed genes during hCMV infection, the inventors developed a reliable assay to predict intrauterine-transmission of hCMV during early stages of gestation. This assay based on measured specific genes expression changes followed by a mathematical analysis that was proved trustworthy by retrospectively identification of individuals conferring risk of congenital virus transmission. Thus, indicating the high probability toward symptomatic neonates, and hence children suffering from sequelae, such as sensorineural hearing loss, cognitive defects, and motor defects.

Specifically, the inventors defined a small set of biomarkers belonging to the IFN-IFIT, OAS, IFI and UBIQUITIN pathway for predicting congenital transmission of hCMV in pregnant women. As shown in the Examples herein, the inventors have found that the expression of ISG15, IFIT3, USP18, EIF2AK2, HERC5, RSAD2 and MX1 can serve to differentiate and predict with high level of confidence (over 94%), if a disease is delivered to a neonates, namely if pregnant women will transmit the disease to their fetus. The biomarkers were derived from PBMC of the pregnant women during the weeks 2-22 of their pregnancy. It was found by the inventors that low expression of ISG15, IFIT3, USP18, EIF2AK2, HERC5, RSAD2 and MX1 is found to correlate with women who have not transmitted the disease, whereas high expression of ISG15, IFIT3, USP18, EIF2AK2, HERC5, RSAD2 and MX1 was found to correlate with women who have transmitted the disease.

These results are in accordance with the mathematical model shown and described herein. The model points out that at the initial entry of the virus, the speed of the IFN fighting genes up regulation should out paste the proteins generated by the virus using the cells resources, for preventing later transfer of the virus to the flatus. The inventors have therefore concluded that the identified genes described herein are suitable for predicting, assessing and monitoring transmission of hCMV to fetus.

Thus, in a first aspect, the invention relates to a method for the prediction, monitoring, and early diagnosis of a viral pathogen transmission, specifically, intrauterine-transmission during early stages of gestation. In more specific embodiments, the method of the invention may comprise the following steps: In a first step (a), determining the level of expression of at least one of ISG15 ubiquitin-like modifier (ISG15), Interferon-induced protein with tetratricopeptide repeats 3 (IFIT3), Ubiquitin specific peptidase 18 (USP18), Eukaryotic Translation Initiation Factor 2 Alpha Kinase 2 (EIF2AK2), HECT And RLD Domain Containing E3 Ubiquitin Protein Ligase 5 (HERC5), Radical S-Adenosyl Methionine Domain Containing 2 (RSAD2) and MX Dynamin Like GTPase 1 (MX1) genes in a biological sample of the subject, to obtain an expression value Exₛₐₘₚ in the sample. The next step (b) involves calculating the M value of the sample (Mₛₐₘₚ). It should be noted that the M value of the sample indicates the ability of the examined subject to prevent intrauterine-transmission of a viral pathogen. In the next step (c), providing a standard M (M_{stand}) value of non-transmitting subjects. It should be noted that the standard M value indicates the minimal ability required for preventing intrauterine-transmission of the viral pathogen. The final step (d) involves determining if the M value of the sample (Mₛₐₘₚ) calculated in step (b) is any one of positive or negative with respect to the standard M (M_{stand}) value of non-transmitting subjects provided in (c). Thus, wherein a positive value of Mₛₐₘₚ indicates that the subject is a non-transmitting subject and a negative value of Mₛₐₘₚ indicates that the subject is a viral pathogen transmitting subject, thereby predicting intrauterine-transmission of a viral pathogen in the subject.

In some embodiments, the method of the invention may comprise in a first step (a), determining the level of expression of at least one of ISG15, IFIT3 and USP18. In some other embodiments, the method of the invention comprise in a first step (a), determining the level of expression of ISG15. In yet some further alternative embodiments, the method comprises in step (a) the determination of the expression of ISG15, IFIT3, USP18, EIF2AK2, HERC5, RSAD2 and MX1 genes. In yet some further embodiments, the method of the invention may comprise in a first step (a), determining the level of expression of ISG15, IFIT3, USP18. In certain embodiments, the method of the invention may comprise in a first step (a), determining the level of expression of EIF2AK2, HERC5, RSAD2 and MX1 genes. It should be appreciated that any of the above-identified genes of the invention may be combined with any of the marker genes disclosed herein, as well as with any gene involved in interferon and/or ubiquitin pathway marker genes. In some specific and non-limiting embodiments, such IFN-related genes may include but are not limited to at least one of ADAR, IFIT1, IFIT2, IFIT3, IFIT5, IRF3, IRF7, IRF9, STAT1, STAT2, IFNAR1, MX1, ISG15 IFNAR2, JAK1, TYK2, EGR1, IFI16, IFI6, XAF1, RNASEL, ISG20, PSMB8, ISGF3, IFI35, RSAD2, OAS1, OAS2, OAS3, OASL, IFITM1, IFITM2, IFITM3, GPB2, MX2, IPGK2, IFI44, IFI44L, IFI27, DDX58, TLR3, TLR7, TLR9, DDX58, DHX58, IFIH1, MAVS and DDX60. In some specific embodiments, the methods, compositions and kits of the invention may further use at least one of said IFN-related genes as additional biomarker/s. In still further embodiments, the ubiquitin pathway related genes may include ISG15, USP18, UBE2L6, HERC5, UBE2E1, MHC-TYPE1:HLA-A,B,C,E, and therefore, at least one of said genes may be used by the methods, compositions and kits of the invention as additional marker genes.

The method provided by the invention is directed to prediction and early diagnosis of intrauterine-transmission of viral pathogens. The term “intrauterine-transmission” or “a vertically transmitted infection” as used herein (or mother-to-child transmission) is an infection caused by bacteria, viruses, or in rare cases, parasites transmitted directly from the mother to an embryo, fetus, or baby during pregnancy or childbirth. This may occur when the mother gets an infection as an intercurrent disease in pregnancy.

In some embodiments, the methods, as well as the compositions and kits of the invention described herein after, may be applicable in detecting, diagnosing and predicting “congenital infection”. In some embodiments the term congenital infection can be used if the vertically transmitted infection persists after childbirth.

Still further, in more specific embodiments calculating the M value of the sample (Mₛₐₘₚ) may be performed by the steps of: (a) obtaining the expression value Exₛₐₘₚ of said sample as determined by the method of the invention; (b) providing a standard curve of expression values of viral pathogen infected subjects; (c) obtaining a maximal expression value Exₘₐₓ and a minimal expression value Exₘᵢₙ from said standard curve of (b); and (d) calculating the Mₛₐₘₚ value of said sample, wherein Mₛₐₘₚ=1−[(Exₛₐₘₚ−Exₘᵢₙ)/(Exₘₐₓ−Exₘᵢₙ)].

In yet another alternative and identical embodiment, the values of Mₛₐₘₚ may be calculated using the equivalent equation:

Mₛₐₘₚ=(Exₘₐₓ−Exₛₐₘₚ)/(Exₘₐₓ−Exₘᵢₙ)].

In some specific embodiments, as also described in Example 2, for an hCMV having a K of 1.92, the standard M required as calculated by the following equation: M=1−1/K, is 0.495 (1−1/1.92). Therefore, any calculated M value of a sample that is greater (a “positive”) value than the standard M, specifically, above 0.495, clearly indicates that the subject is a non-transmitting subject. In a similar manner, any calculated Mₛₐₘₚ that is below 0.495 (a “negative” value) indicates that the subject is hCMV transmitting subject.

Thus, in some specific embodiments, the method of the invention is specifically applicable for detecting and predicting intrauterine-transmission of a viral pathogen that may be a human cytomegalovirus (hCMV).

In some specific embodiments the method of the invention may be applicable for a human female subject. In more specific embodiments, the method of the invention may be applicable for a pregnant human female.

In further embodiments, the pregnant human female subject may be at early stage of gestation. As used herein Gestation is the carrying of an embryo or fetus inside female viviparous animals. It is typical for mammals, but also occurs for some non-mammals. Mammals during pregnancy can have one or more gestations at the same time (multiple gestations).

The time interval of a gestation is called the gestation period. In human obstetrics, gestational age refers to the embryonic or fetal age plus two weeks. This is approximately the duration since the woman's last menstrual period (LMP) began. Human pregnancy can be divided roughly into three trimesters, each approximately three months long. In humans, birth normally occurs at a gestational age of about 40 weeks, though it is common for births to occur from 37 to 42 weeks. After 8 weeks, the embryo is called a fetus.

More specifically, Gestational age (or menstrual age) is a measure of the age of a pregnancy where the origin is the woman's last normal menstrual period (LMP), or the corresponding age as estimated by other methods. Such methods include adding 14 days to a known duration since fertilization (as is possible in in vitro fertilization), or by obstetric ultrasonography. The popularity of using such a definition of gestational age is that menstrual periods are essentially always noticed, while there is usually a lack of a convenient way to discern when fertilization occurred.

The initiation of pregnancy for the calculation of gestational age or stage can be different from definitions of initiation of pregnancy in context of the abortion debate or beginning of human personhood.

As noted above, stages of gestation may refers to three main stages, also referred to as trimester. The first three-month trimester is generally calculated as starting on the first day of the last period and runs through the 13^{th} week of the pregnancy. During this trimester, the fetus implants into the womb of the woman.

The second three-month trimester starts in the 14^{th} week of the pregnancy and runs through the 27^{th} week. During the 2^{nd} trimester.

The third three-month trimester starts in the 28^{th} week of e gestation period and runs through the birth of the child. During this period, the fetus increases in size and stretches the uterus and abdomen of the woman.

In some specific embodiments, the methods of the invention ay be applicable for any stage of gestation. In some specific embodiments, the method may be applicable to early stage of gestation. Early stage of gestation refers to in some embodiments to first and second trimesters, specifically, weeks 2 to 22 of the pregnancy, more specifically, week 2, 3, 4, 5, 6, 7, 8, 9, 10, 11, 12, 1.3, 14, 15, 16, 17, 18, 19, 20, 21, 22 and more.

Still further, determining the level of expression of at least one of said ISG15, IFIT3, USP18, EIF2AK2, HERC5, RSAD2 and MX1 genes in a biological sample of the subject may be performed by the step of contacting detecting molecules specific for said genes with a biological sample of said subject, or with any nucleic acid or protein product obtained therefrom.

More specifically, determining the level of expression of at least one of said ISG15, IFIT3, USP18, EIF2AK2, HERC5, RSAD2 and MX1 biomarker genes is performed by the step of contacting at least one detecting molecule or any combination or mixture of plurality of detecting molecules with a biological sample of said subject, or with any gene or nucleic acid product obtained therefrom, wherein each of said detecting molecules is specific for one of said biomarker genes. The term “contacting” mean to bring, put, incubates or mix together. As such, a first item is contacted with a second item when the two items are brought or put together, e.g., by touching them to each other or combining them. In the context of the present invention, the term “contacting” includes all measures or steps which allow interaction between the at least one of the detection molecules of at least one of the biomarker genes, and optionally, for at least one suitable control reference gene of the tested sample. The contacting is performed in a manner so that the at least one of detecting molecule of at least one of the biomarker genes for example, can interact with or bind to the at least one of the biomarker genes, in the tested sample. The binding will preferably be non-covalent, reversible binding, e.g., binding via salt bridges, hydrogen bonds, hydrophobic interactions or a combination thereof.

In more specific embodiments, the detecting molecules may be selected from isolated detecting nucleic acid molecules and isolated detecting amino acid molecules.

In yet some further embodiments, the nucleic acid detecting molecule comprises isolated oligonucleotide/s, each oligonucleotide specifically hybridizes to a nucleic acid sequence of said at least one of ISG15, IFIT3, USP18, EIF2AK2, HERC5, RSAD2 and MX1 genes and optionally, to a control reference gene.

In more specific embodiments the detecting molecule may be at least one of a pair of primers, at least one primer, nucleotide probes or any combinations thereof. According to certain embodiments, the sample examined by the method of the invention may be any one of peripheral blood mononuclear cells, amniotic fluid and biopsies of organs or tissues.

Still further, according to other embodiments, the method of the invention uses any appropriate biological sample. The term “biological sample” in the present specification and claims is meant to include samples obtained from a mammal subject.

It should be recognized that in certain embodiments a biological sample may be for example, amniotic fluid, bone marrow, lymph fluid, blood cells, blood, serum, plasma, urine, sputum, saliva, faeces, semen, spinal fluid or CSF, the external secretions of the skin, respiratory, intestinal, and genitourinary tracts, tears, milk, any human organ or tissue, any sample obtained by lavage, optionally of the breast ducal system, plural effusion, sample of in vitro or ex vivo cell culture and cell culture constituents. More specific embodiments, the sample may be any one of peripheral blood mononuclear cells and amniotic fluid.

According to some embodiments of the invention, the sample is a cell sample. More specifically, the cell is a blood cell (e.g., white blood cells, macrophages, B- and T-lymphocytes, monocytes, neutrophiles, eosinophiles, and basophiles) which can be obtained using a syringe needle from a vein of the subject. It should be noted that the cell may be isolated from the subject (e.g., for in vitro detection) or may optionally comprise a cell that has not been physically removed from the subject (e.g., in vivo detection).

According to a specific embodiment, the sample used by the method of the invention is a sample of peripheral blood mononuclear cells (PBMCs).

The phrase, “peripheral blood mononuclear cells (PBMCs)” as used herein, refers to a mixture of monocytes and lymphocytes. Several methods for isolating white blood cells are known in the art. For example, PBMCs can be isolated from whole blood samples using density gradient centrifugation procedures. Typically, anticoagulated whole blood is layered over the separating medium. At the end of the centrifugation step, the following layers are visually observed from top to bottom: plasma/platelets, PBMCs, separating medium and erythrocytes/granulocytes. The PBMC layer is then removed and washed to remove contaminants (e.g., red blood cells) prior to determining the expression level of the polynucleotide (s) bio-markers of the invention.

In yet another embodiment, the sample may be amniotic fluid.

The amniotic fluid, commonly called a pregnant woman's water or waters (Latin liquor amnii), is the protective liquid contained by the amniotic sac or gestational sac of a pregnant female Amniotic fluid is present from the formation of the gestational sac or the amniotic sac. It is generated from maternal plasma, and passes through the fetal membranes by osmotic and hydrostatic forces. When fetal kidneys begin to function in about week 16, fetal urine also contributes to the fluid. The fluid is absorbed through the fetal tissue and skin. After the 20th-25th week of pregnancy when the keratinization of an embryo's skin occurs, the fluid is primarily absorbed by the fetal gut. At first, amniotic fluid mainly comprise water with electrolytes, but by about the 12-14th week the liquid also contains proteins, carbohydrates, lipids and phospholipids, and urea, all of which aid in the growth of the fetus. The volume of amniotic fluid increases with the growth of fetus. From the 10^{th} to the 20th week it increases from 25 ml to 400 ml approximately and is about 1 liter at birth. Amniotic fluid normally has a pH of 7.0 to 7.5.

As noted above, the invention provides prognostic and predictive methods for identifying subjects that are likely to intrauterine-transmit viral pathogens, specifically, CMV to the embryo. “Prognosis” is defined as a forecast of the future course of a condition, based on medical knowledge. This highlights the major advantage of the invention, namely, the ability to predict transmission of the viral pathogen to the embryo, based on the expression value of at least one of the biomarker genes of the invention and the predictive methods and M values calculated therefrom. More specifically, the ability to determine at early stage that the subject may transmit CMV to fetus.

The methods, compositions and kits of the invention provide early diagnosis and prediction of transmission of viral pathogens, specifically, CMV. An “early diagnosis” provides diagnosis prior to appearance of clinical symptoms. Prior as used herein is meant days, weeks, months or even years before the appearance of such symptoms. In more specific embodiments, the methods, compositions and kits of the invention provide prediction of transmission risk of a subject before infection. Specifically, the method provides a prediction of non-pregnant and non-infected subjects, estimating the ability of the examined subject to avoid or prevent intrauterine-transmission f viral pathogens, specifically, CMV. More specifically, at least 1 week, at least 1 month, 2 months, 3 months, 4 months, 5 months, 6 months, 7 months, 8 months, 9 months, 10 months, 11 months, 12 months, or even few years before at least one of pregnancy, infection or clinical symptoms appear.

In some embodiments, the methods of the invention may provide prediction of transmission of the virus, before the appearance of any of the clinical symptoms discussed in the present specification, specifically, any of the clinical symptoms associated with CMV infection.

In yet some further specific embodiments the diagnostic and predictive methods of the invention may combine a predictive method with some therapeutic and prophylactic applications. Therefore, in certain embodiments the method of the invention may further comprise the step of informing, notifying or consulting a subject predicted or diagnosed as intrauterine-transmitting a viral pathogen, specifically, hCMV, or a subject that is predicted to transmit viral pathogen to an embryo. In yet some further embodiments, the methods of the invention may comprise an additional step of recommending, applying or suggesting further therapeutic steps necessary to prevent manifestation of embryonic damage caused by said viral pathogen, specifically, CMV. These further therapeutic and prophylactic steps may include but are not limited to abortion, anti-viral treatment and the like.

Thus, in some further embodiments, the invention provides a prophylactic method for preventing and reducing viral-pathogen associated disorders, specifically, CMV-related disorders.

As noted herein before, congenital cytomegalovirus (CMV) infection is a major public health concern. CMV causes serious neurodevelopmental sequelae, including mental retardation, cerebral palsy, and sensorineural hearing loss (SNHL). Even with antiviral therapy, these injuries are often irreversible. The pathogenesis of injury to the developing fetal central nervous system (CNS) is unknown.

Specifically, congenital CMV infection is the major cause of birth defects and childhood disorders in the United States. It is estimated that about 40,000 children (0.2 to 2% of all deliveries) are born with CMV, resulting in about 400 fatal cases each year. Only 10 to 15% of children with congenital CMV infection exhibit clinical signs at birth, although even children who appear asymptomatic at birth are at risk for neurodevelopmental sequelae. Most children (60 to 90%) with symptomatic infection, and 10 to 15% of those with asymptomatic infection, develop one or more long-term neurological sequelae, such as mental retardation, psychomotor retardation, SNHL, and ophthalmologic abnormalities. Current estimates indicate that approximately 8,000 children are affected each year with some neurological sequelae related to in utero CMV infection.

More specifically, in utero infection is believed to be due to maternal viremia with attendant hematogenous spread to the fetus. The rate of materno-fetal transmission is influenced by numerous factors, including trimester of exposure, maternal age, CMV serostatus, character of maternal immunity, and viral loads. The risk of fetal transmission appears to increase with gestational age, but neurological outcomes are more severe when infection occurs during the first trimester. However, viral transmission can occur during the entire gestation period, and neurological outcomes may still be seen from infections acquired in late gestation.

Among the primary clinical manifestations associated with congenital CMV infection, the most devastating are those involving the developing CNS, since in contrast to other end-organ injury, CNS injury is generally believed to be irreversible. The most commonly observed symptoms of CMV infection at birth are intrauterine growth retardation, purpura, jaundice, hepatosplenomegaly, microencephaly, hearing impairment, and thrombocytopenia. While clinical signs due to abnormalities of the reticuloendothelial system (such as anemia, hepatosplenomegaly, and jaundice) are transient, neurological deficits either are evident at birth and typically persist for life or tend to become evident (as SNHL) in early childhood.

Commonly seen are chronic lesions due to infection, which include ventricular dilatation, white matter gliosis, atrophy (volume loss), parenchymal cysts, ependymal cysts, calcifications, and cortical malformations (most notably polymicrogyria). Periventricular cysts develop during the second trimester, cerebellar lesions probably are the result of fetal infection before 18 weeks of gestation, and abnormal sulcations probably are due to injury between 18 and 24 weeks.

Other abnormalities observed in the spectrum of neuroimaging/pathological abnormalities include lissencephaly, porencephaly, and schizencephaly.

CMV-induced hearing loss is believed to be caused by virus-induced labyrinthitis. Inner ear histology from congenitally infected infants shows damage to structures including the vestibular endolymphatic system and the vestibular organs (saccule and utricle) and collapse of the saccular membrane. Damage is restricted to the endolymphatic structures, with minor involvement of the cochlea, manifest mainly as hydrops at the basal turn.

A second aspect of the invention relates to a kit comprising detecting molecules specific for determining the level of expression of ISG15, IFIT3 and USP18, genes in a biological sample.

In some specific embodiments, the kit of the invention may further comprise detecting molecules specific for determining the level of expression of at least one of, EIF2AK2, HERC5, RSAD2 and MX1. In yet some further embodiments, the kit of the invention may comprise detecting molecules specific for determining the level of expression of ISG15, IFIT3, USP18, EIF2AK2, HERC5, RSAD2 and MX1.

In some specific embodiments, the kit of the invention may further comprise at least one of: (a) means for calculating the M value of a tested subject (Mₛₐₘₚ) It should be noted that such value indicates the ability of the examined subject to prevent intrauterine-transmission of a viral pathogen. (b) a standard M (M_{stand}) value of non-transmitting subjects. It should be noted that the M_{stand} value indicates the minimal ability required for preventing intrauterine-transmission of said viral pathogen. The kit may further comprise (c) means for calculating a standard M_{stand} value for non-transmitting population; (d) detecting molecules specific for determining the level of expression of at least one control reference gene in a biological sample; and (e) at least one control sample.

In some specific embodiments, means for calculating the value of M of a tested subject (Mₛₐₘₚ) may comprise at least one of:

(a) detecting molecules specific for determining the level of expression of ISG15, IFIT3, USP18 and optionally, EIF2AK2, HERC5, RSAD2 and MX1 genes in a biological sample for determining an expression value Exₛₐₘₚ in said sample;
(b) pre-determined calibration curve providing standard expression values of ISG15, IFIT3 USP18, and optionally, EIF2AK2, HERC5, RSAD2 and MX1 genes in viral pathogen infected subjects or predetermined maximal expression value Exₘₐₓ and a minimal expression value Exₘᵢₙ calculated from said standard curve; and
(c) a formula for calculating Mₛₐₘₚ value, wherein said formula is Mₛₐₘₚ=[(Exₛₐₘₚ−Exₘᵢₙ)/(Exₘₐₓ−Exₘᵢₙ)].

In some specific embodiments, the viral pathogen may be hCMV. Thus, in certain embodiments the kit of the invention may be used as a diagnostic kit for predicting intrauterine-transmission of hCMV in a mammalian subject.

In some embodiments, the subject is an hCMV infected subject. More specifically, the subject may be a human female subject.

In some specific embodiments, the pregnant human female subject may be pregnant at early stage of gestation. In yet some further embodiments, the female subject may be a non-infected subject. In some embodiments, the subject may be pregnant and non-infected, in some further embodiments, the subject may be non-pregnant and non-infected, non-pregnant and infected or pregnant and infected subject.

The invention therefore provides methods, compositions and kits for predicting intrauterine-transmission of a viral pathogen, even before infection and/or pregnancy occur, and therefore reflect the ability of a specific subject to avoid or prevent intrauterine-transmission of a viral pathogen.

In certain embodiments the kit of the invention may further comprise instructions for use, wherein said instructions comprise at least one of:

(a) instructions for carrying out the detection and quantification of expression of said ISG15, IFIT3, USP18, and optionally EIF2AK2, HERC5, RSAD2 and MX1 genes;
(b) instructions for carrying out the detection and quantification of expression of said at least one control reference gene; and
(c) instructions for determining if the calculated M value of said sample (Mₛₐₘₚ) is any one of positive or negative with respect to the standard M (M_{stand}) value of non-transmitting subjects.

In certain embodiments, the detection step further involves detecting a signal from the detecting molecules that correlates with the expression level of at least one of the biomarker genes and in the sample from the subject, by a suitable means. According to some embodiments, the signal detected from the sample by any one of the experimental methods detailed herein below reflects the expression level of at least one of the biomarker genes. It should be noted that such signal-to-expression level data may be calculated and derived from a calibration curve.

Thus, in certain embodiments, the methods, compositions and kits of the invention may optionally further involve the use of a calibration curve created by detecting a signal for each one of increasing pre-determined concentrations of at least one of the biomarker genes. Obtaining such a calibration curve may be indicative to evaluate the range at which the expression levels correlate linearly with the concentrations of at least one of the biomarker genes. It should be noted in this connection that at times when no change in expression level of at least one of the biomarker genes is observed, the calibration curve should be evaluated in order to rule out the possibility that the measured expression level is not exhibiting a saturation type curve, namely a range at which increasing concentrations exhibit the same signal.

It must be appreciated that in certain embodiments such calibration curve as described above may by also part or component in any of the kits provided by the invention.

In some embodiments the detecting molecules comprise at least one of isolated detecting nucleic acid molecules and isolated detecting amino acid molecules.

In further embodiments, the detecting molecules comprise isolated oligonucleotides, each said oligonucleotide specifically hybridize to a nucleic acid sequence of an RNA product of one of said ISG15, IFIT3, USP18, any optionally, EIF2AK2, HERC5, RSAD2 and MX1 genes.

In some specific embodiments, the detecting molecules may be at least one of at least one primer, at least one pair of primers, at least one nucleotide probe and any combination thereof.

In certain embodiments, the kit of the invention may further comprise at least one reagent for conducting a nucleic acid amplification based assay selected from the group consisting of a Real-Time PCR, micro arrays, PCR, in situ Hybridization and Comparative Genomic Hybridization.

In some specific embodiments, the kit of the invention may further comprise a solid support, wherein each of said detecting molecules is disposed in an array.

In more specific embodiments, the array of detecting molecules may comprise a plurality of addressed vessels.

Still further, the array of detecting molecules may comprise a solid support holding detecting molecules in distinct regions.

In certain embodiments, the kit of the invention may be applicable for samples that may be at least one of a blood sample and amniotic fluid.

In some specific embodiments, the sample is a blood sample, and the kit comprises detecting molecule/s specific for determining the level of expression of ISG15, IFIT3, USP18, and optionally, EIF2AK2, HERC5, RSAD2 and MX1 genes in said blood sample.

The invention further provides an array of detecting molecules specific for ISG15, IFIT3 and USP18, genes. More specifically, such detecting molecules may be isolated detecting nucleic acid molecules or isolated detecting amino acid molecule/s.

In some specific embodiments, the array may further comprise detecting molecules specific for EIF2AK2, HERC5, RSAD2 and MX1 genes.

In some embodiments, the array of the invention may further comprise a plurality of addressed vessels containing said detecting molecule/s.

In certain embodiments, the array of the invention may comprise a solid support holding detecting molecules in distinct regions.

In a further aspect, the invention provides a prognostic composition comprising detecting molecules specific for determining the level of expression of ISG15, IFIT3 and USP18, genes in a biological sample.

In yet some further embodiments, the diagnostic composition of the invention may further comprise detecting molecules specific for determining the level of expression of EIF2AK2, HERC5, RSAD2 and MX1 genes in a biological sample.

In certain embodiments, such prognostic composition may be applicable for the prediction and diagnosis of intrauterine-transmission of a viral pathogen, specifically, human cytomegalovirus (hCMV) in a mammalian subject.

As mentioned above, the method and kits of the invention may use the marker genes provided herein, specifically, any one of or at least one of ISG15, IFIT3, USP18, EIF2AK2, HERC5, RSAD2 and MX1, any combinations thereof and any combinations thereof with any additional biomarker genes.

In some embodiments, the methods, compositions and kits of the invention may use ISG15 as a marker gene. More specifically, ISG15 ubiquitin-like modifier (ISG15) gene (GenBank Accession No. NM_005101; SEQ ID NO: 1) encodes the ISG15 protein (GenBank Accession No. NP_005092.1; SEQ ID NO: 2). ISG15 is reported to be an ubiquitin-like protein that is conjugated to intracellular target proteins after IFN-alpha or IFN-beta stimulation. Its enzymatic pathway is partially distinct from that of ubiquitin, differing in substrate specificity and interaction with ligating enzymes. ISG15 conjugation pathway uses a dedicated E1 enzyme, but seems to converge with the ubiquitin conjugation pathway at the level of a specific E2 enzyme. Targets include STAT1, SERPINA3G/SPI2A, JAK1, MAPK3/ERK1, PLCG1, EIF2AK2/PKR, MX1/MxA, and RIG-1. It undergoes deconjugation by USP18/UBP43. It shows specific chemotactic activity towards neutrophils and activates themu to induce release of eosinophil chemotactic factors. It was suggested to serve as a trans-acting binding factor directing the association of ligated target proteins to intermediate filaments.

In yet some further embodiments, the methods, compositions and kits of the invention may use IFIT3 as a marker gene. Interferon-induced protein with tetratricopeptide repeats 3 (IFIT3) gene (GenBank Accession Nos. NM_001031683; SEQ ID NO: 3, NM_001549; SEQ ID NO: 4) encodes the IFIT3 protein (GenBank Accession Nos. NP_001026853; SEQ ID NO: 5, NP_001540; SEQ ID NO: 6).

In still further embodiments, the methods, compositions and kits of the invention may use USP18 as a marker gene. Ubiquitin specific peptidase 18 (USP18) gene (GenBank Accession No. MN_017414; SEQ ID NO: 7) encodes the USP18 protein (GenBank Accession No. NP_059110 SEQ ID NO: 8). The protein encoded by this gene belongs to the ubiquitin-specific proteases (UBP) family of enzymes that cleave ubiquitin from ubiquitinated protein substrates. It is highly expressed in liver and thymus, and is localized to the nucleus. USP18 protein efficiently cleaves only ISG15 (a ubiquitin-like protein) fusions, and deletion of this gene in mice results in a massive increase of ISG15 conjugates in tissues, indicating that this protein is a major ISG15-specific protease. Mice lacking this gene are also hypersensitive to interferon, suggesting a function of this protein in down regulating interferon responses, independent of its isopeptidase activity towards ISG15. USP18 can efficiently cleave only ISG15 fusions including native ISG15 conjugates linked via isopeptide bonds. Necessary to maintain a critical cellular balance of ISG15-conjugated proteins in both healthy and stressed organisms.

In yet some further embodiments, the methods, compositions and kits of the invention may use EIF2AK2 as a marker gene. Eukaryotic Translation Initiation Factor 2 Alpha Kinase 2 (EIF2AK2) gene (GenBank Accession No. NM_001135651.2; SEQ ID NO: 9, NM_001135652.2; SEQ ID NO:10, NM_002759.3, SEQ ID NO: 11) respectively encodes the ISG15 protein (GenBank Accession No. NP_001129123; SEQ ID NO:12, NP_001129124; SEQ ID NO:13, NP_002750; SEQ ID NO:14). EIF2AK2 is a serine/threonine protein kinase that is activated by autophosphorylation after binding to dsRNA. The activated form of the encoded protein can phosphorylate translation initiation factor EIF2S1, which in turn inhibits protein synthesis. EIF2AK2 is also activated by manganese ions and heparin.

In further embodiments, the methods, compositions and kits of the invention may use HERC5 as a marker gene. HECT and RLD domain containing E3 ubiquitin protein ligase 5 (HERC5) gene (GenBank Accession No. NM_016323; SEQ ID NO: 15) encodes the HERC5 protein (GenBank Accession No. NP_057407 SEQ ID NO: 16). HERC5 gene is a member of the HERC family of ubiquitin ligases and encodes a protein with a HECT domain and five RCC1 repeats. Pro-inflammatory cytokines up regulate expression of this gene in endothelial cells. The HERC5 protein localizes to the cytoplasm and perinuclear region and functions as an interferon-induced E3 protein ligase that mediates ISGylation of protein targets. It is a major E3 ligase for ISG15 conjugation. HERC5 Acts as a positive regulator of innate antiviral response in cells induced by interferon. Makes part of the ISGylation machinery that recognizes target proteins in a broad and relatively non-specific manner.

In yet some further embodiments, the methods, compositions and kits of the invention may use RSAD2 as a marker gene. Radical S-adenosyl methionine domain containing 2 (RSAD2) gene (GenBank Accession No. NM_080657; SEQ ID NO:17) encodes the RSAD2 protein (GenBank Accession No. NP_542388; SEQ ID NO: 18). RSAD2 is reported to be involved in antiviral defense. It was suggested to impair virus budding by disrupting lipid rafts at the plasma membrane, a feature which is essential for the budding process of many viruses. In addition, it was reported to act through binding with and inactivating FPPS, an enzyme involved in synthesis of cholesterol, farnesylated and geranylated proteins, ubiquinones dolichol and heme. Moreover, it is considered to play a major role in the cell antiviral state induced by type I and type II interferon. Finally, it was reported to display antiviral effect against HIV-1 virus, hepatitis C virus, human cytomegalovirus, and aphaviruses, but not vesiculovirus.

In further embodiments, the methods, compositions and kits of the invention may use MX1 as a marker gene. Myxovirus (influenza virus) resistance 1 (MX1) gene (GenBank Accession No. NM_002462 SEQ ID NO:19, NM_001178046 SEQ ID NO:21, NM_001144925 SEQ ID NO:23) respectively encodes the MX1 protein (GenBank Accession No. NP_002453 SEQ ID NO:20, NP_001171517 SEQ ID NO:22, NP_001138397 SEQ ID NO:24). In mouse, the interferon-inducible Mx protein is responsible for a specific antiviral state against influenza virus infection. The protein encoded by this gene is similar to the mouse protein as determined by its antigenic relatedness, induction conditions, physicochemical properties, and amino acid analysis. This cytoplasmic protein is a member of both the dynamin family and the family of large GTPases. Two transcript variants encoding the same protein have been found for this gene. MX1 may regulate the calcium channel activity of TRPCs. Ring-like assemblies may induce membrane tabulation.

In some embodiments, the methods, compositions and kits of the invention may use ISG15, IFIT3, USP18, EIF2AK2, HERC5, RSAD2 and MX1 as the marker genes. In some alternative embodiments, the methods, compositions and kits of the invention may use ISG15, IFIT3 and USP18 as the marker genes. Still further, the methods, compositions and kits of the invention may use EIF2AK2, HERC5, RSAD2 and MX1 as the marker genes.

In yet some further embodiments, it must be appreciated that the methods and kits of the invention may use further marker genes. Non-limiting examples for such additional genes may include at least one of IFIT1 and OAS2.

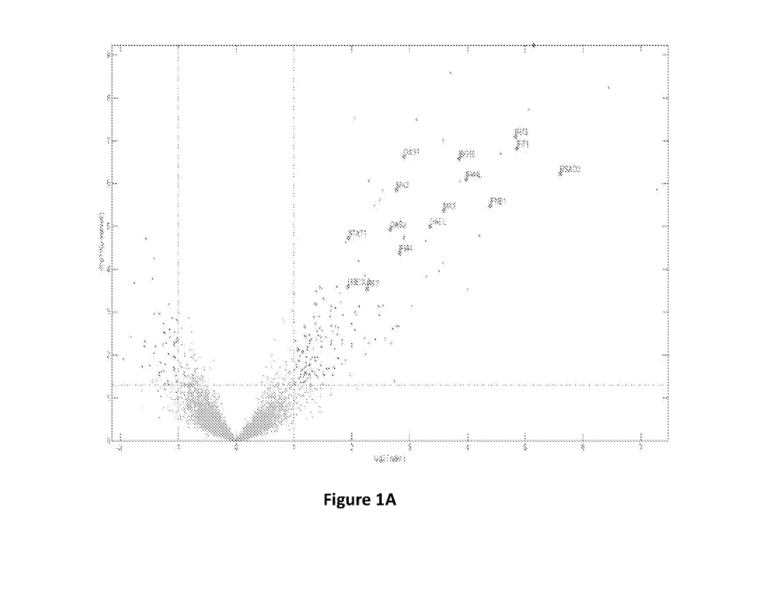

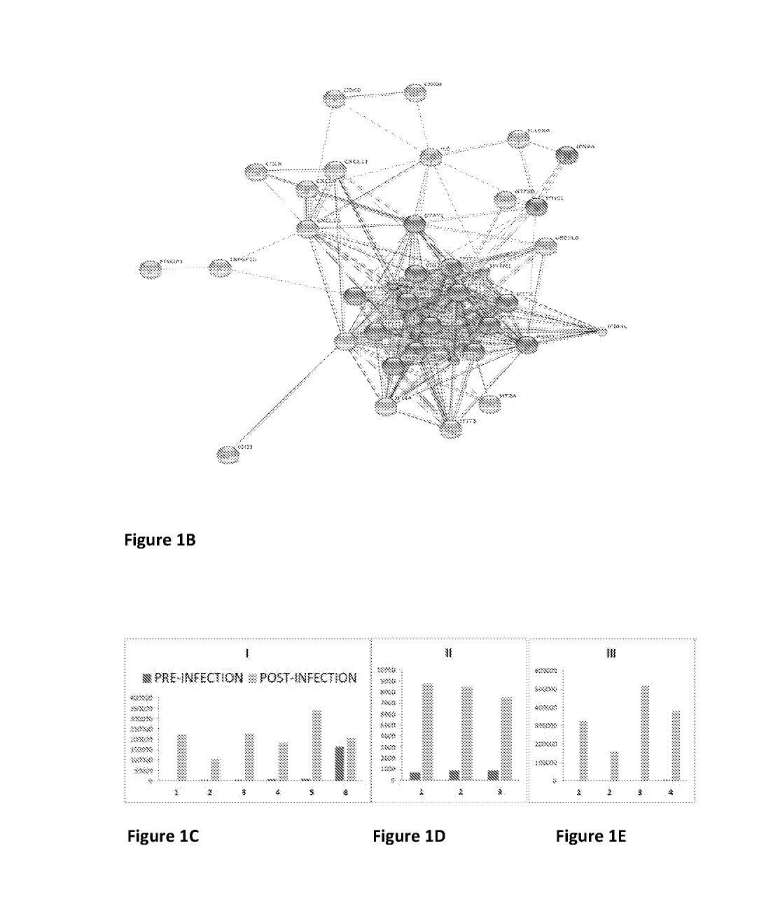

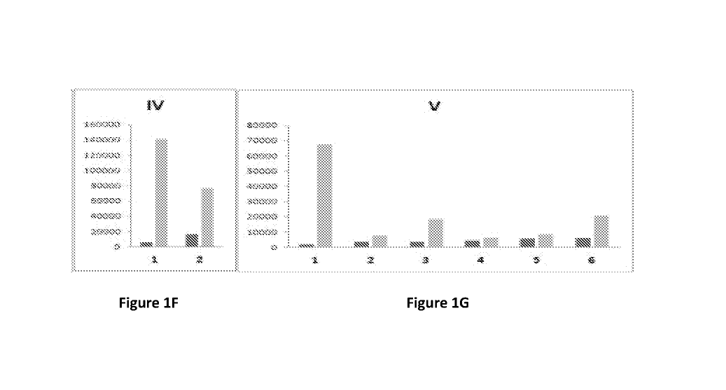

In yet some further embodiments, the additional gene/s may be any of the marker genes disclosed by the invention, in some specific embodiments, any of the genes disclosed in FIGS. 1A and/or 1B. Still further, in certain embodiments, any gene participating in at least one of ubiquitin or interferon pathways, may be used as an additional marker in the methods, kits and compositions of the invention. In some specific and non-limiting embodiments, such IFN-related genes may include but are not limited to at least one of ADAR, IFIT1, IFIT2, IFIT3, IFIT5, IRF3, IRF7, IRF9, STAT1, STAT2, IFNAR1, MX1, ISG15 IFNAR2, JAK1, TYK2, EGR1, IFI16, IFI6, XAF1, RNASEL, ISG20, PSMB8, ISGF3, IFI35, RSAD2, OAS1, OAS2, OAS3, OASL, IFITM1, IFITM2, IFITM3, GPB2, MX2, IPGK2, IFI44, IFI44L, IFI27, DDX58, TLR3, TLR7, TLR9, DDX58, DHX58, IFIH1, MAVS and DDX60. In some specific embodiments, the methods, compositions and kits of the invention may further use at least one of said IFN-related genes as additional biomarker/s. In still further embodiments, the ubiquitin pathway related genes may include ISG15, USP18, UBE2L6, HERC5, UBE2E1, MHC-TYPE1:HLA-A,B,C,E, and therefore, at least one of said genes may be used by the methods, compositions and kits of the invention as additional marker genes.

It should be noted that each detecting molecule used by the method of the invention is specific for one biomarker. In some embodiments, the method as well as the methods, compositions and kits of the invention described herein after may provide and use further detecting molecules specific for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more, specifically, 110, 120, 130, 140, 150, 160, 170, 180, 190, 200, 250, 300, 350, 400, 450 and 500 at the most, additional biomarker genes. In some embodiments, the methods, compositions and kits of the invention may provide and use in addition to detecting molecules specific for at least one of the biomarkers genes of the invention, specifically, at least one of ISG15 ubiquitin-like modifier (ISG15), Interferon-induced protein with tetratricopeptide repeats 3 (IFIT3), Ubiquitin specific peptidase 18 (USP18), Eukaryotic Translation Initiation Factor 2 Alpha Kinase 2 (EIF2AK2), HECT And RLD Domain Containing E3 Ubiquitin Protein Ligase 5 (HERC5), Radical S-Adenosyl Methionine Domain Containing 2 (RSAD2) and MX Dynamin Like GTPase 1 (MX1), also detecting molecule/s specific for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more biomarker genes, and optionally, further detecting molecule/s specific for additional at least one biomarker gene/s. In some embodiments, the methods, as well as the compositions and kits of the invention may provide and use detecting molecules specific for at least one additional biomarker gene and at most, 499 additional marker gene/s. In some specific embodiments, the methods and kit/s of the invention may provide and use detecting molecules specific for at least one of the biomarker genes of the invention, and detecting molecules specific for at least one additional biomarkers, provided that detecting molecules specific for 100, 150, 200, 250, 300, 350, 384, 400, 450 and 500 at the most biomarker genes are used. In still further specific and non-limiting embodiments, the at least one additional biomarker gene may comprise any of the biomarker genes presented in FIGS. 1a and 1B.

Still further, in certain embodiments, any gene participating in at least one of ubiquitin or interferon pathways, may be used as an additional marker in the methods, kits and compositions of the invention.

In some specific and non-limiting embodiments, such IFN-related genes may include but are not limited to at least one of ADAR, IFIT1, IFIT2, IFIT3, IFIT5, IRF3, IRF7, IRF9, STAT1, STAT2, IFNAR1, MX1, ISG15 IFNAR2, JAK1, TYK2, EGR1, IFI16, IFI6, XAF1, RNASEL, ISG20, PSMB8, ISGF3, IFI35, RSAD2, OAS1, OAS2, OAS3, OASL, IFITM1, IFITM2, IFITM3, GPB2, MX2, IPGK2, IFI44, IFI44L, IFI27, DDX58, TLR3, TLR7, TLR9, DDX58, DHX58, IFIH1, MAVS and DDX60. In some specific embodiments, the methods, compositions and kits of the invention may further use at least one of said IFN-related genes as additional biomarker/s. In still further embodiments, the ubiquitin pathway related genes may include ISG15, USP18, UBE2L6, HERC5, UBE2E1, MHC-TYPE1:HLA-A,B,C,E, and therefore, at least one of said genes may be used by the methods, compositions and kits of the invention as additional marker genes.

In yet some further embodiments, it should be understood that the methods of the invention as well as the compositions and kits described herein after may involve the determination of the expression levels of the biomarker genes of the invention and/or the use of detecting molecules specific for said biomarker genes. Specifically, at least one, at least two, at least three, at least four, at least five, at least six and at least seven, of the biomarker gene/s of the invention that may further comprise any additional biomarker genes or control reference gene provided that 500 at the most biomarker genes and control reference genes are used. In some embodiments, the at least one, at least two, at least three, at least four, at least five, at least six and at least seven of the biomarker gene/s of the invention may form at least about 10%, 15%, 20%, 25%, 30%, 35%, 40%, 45%, 50%, 55%, 60%, 65%, 70%, 75%, 80%, 85%, 90%, 95% or 100% of the biomarker genes determined by the methods of the invention. In yet some further embodiments, the detecting molecules specific for at least one, at least two, at least three, at least four, at least five, at least six and at least seven of the biomarker gene/s of the invention, that are used by the methods of the invention and comprised within any of the compositions and kits of the invention may form at least 10%, 15%, 20%, 25%, 30%, 35%, 40%, 45%, 50%, 55%, 60%, 65%, 70%, 75%, 80%, 85%, 90%, 95% or 100% of detecting molecules used in accordance with the invention. It should be appreciated that for each of the selected biomarker genes at least one detecting molecules may be used. In case more than one detecting molecule is used for a certain biomarker gene, such detecting molecules may be either identical or different.

In more specific embodiments the method of the invention may involves the determination of the expression level of at least 1, 2, 3, 4, 5, 6, 7 or more of the biomarker genes of the invention, specifically, the genes disclosed by the invention, specifically, at least one of ISG15, IFIT3, USP18, EIF2AK2, HERC5, RSAD2 and MX1 genes, and optionally further at least 1, 2, 3, 4, 5, 6, 7, 8, 9, 10, 11, 12, 13, 14, 15, 16, 17, 18, 19, 20, 21, 22, 23, 34, 35, 36, 37, 38, 39, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more of the biomarker genes disclosed by the invention, for examples in any one of FIGS. 1A, 1B disclosed herein or any further marker gene/s. It should be appreciated that further biomarkers may be used, for example, any of the biomarkers disclosed by the invention. In some further embodiments, the method of the invention may involve determination of the expression level of additional biomarker gene/s, specifically, additional at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more, specifically, 110, 120, 130, 140, 150, 160, 170, 180, 190, 200, 250, 300, 350, 400, 450 and at most, 500 additional biomarker genes.

As noted above, the methods and kits of the invention involve determining the expression level of marker genes in a sample. The terms “level of expression” or “expression level” are used interchangeably and generally refer to a numerical representation of the amount (quantity) of a polynucleotide which encodes an amino acid product or protein in a biological sample.

“Expression” generally refers to the process by which gene-encoded information is converted into the structures present and operating in the cell. For example, biomarker gene expression values measured in Real-Time Polymerase Chain Reaction, sometimes also referred to as RT-PCR or quantitative PCR (qPCR), represent luminosity measured in a tested sample, where an intercalating fluorescent dye is integrated into double-stranded DNA products of the qPCR reaction performed on reverse-transcribed sample RNA, i.e., test sample RNA converted into DNA for the purpose of the assay. The luminosity is captured by a detector that converts the signal intensity into a numerical representation which is said expression value, in terms of miRNA. Therefore, according to the invention “expression” of a gene, specifically, a gene encoding the biomarker genes of the invention may refer to transcription into a polynucleotide. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. Methods for determining the level of expression of the biomarkers of the invention will be described in more detail herein after.

In certain and specific embodiments, the step of determining the level of expression to obtain an expression value by the method of the invention further comprises an additional and optional step of normalization. According to this embodiment, in addition to determination of the level of expression of the biomarkers of the invention, the level of expression of at least one suitable control reference gene (e.g., housekeeping genes) is being determined in the same sample. According to such embodiment, the expression level of the biomarkers of the invention obtained in step (a) is normalized according to the expression level of said at least one reference control gene obtained in the additional optional step in said test sample, thereby obtaining a normalized expression value. Optionally, similar normalization is performed also in at least one control sample or a representing standard when applicable.

The term “expression value” thus refers to the result of a calculation, that uses as an input the “level of expression” or “expression level” obtained experimentally and by normalizing the “level of expression” or “expression level” by at least one normalization step as detailed herein, the calculated value termed herein “expression value” is obtained.

More specifically, as used herein, “normalized values” are the quotient of raw expression values of marker genes, divided by the expression value of a control reference gene from the same sample, such as a stably-expressed housekeeping control gene. Any assayed sample may contain more or less biological material than is intended, due to human error and equipment failures. Importantly, the same error or deviation applies to both the marker genes of the invention and to the control reference gene, whose expression is essentially constant. Thus, division of the marker gene raw expression value by the control reference gene raw expression value yields a quotient which is essentially free from any technical failures or inaccuracies (except for major errors which destroy the sample for testing purposes) and constitutes a normalized expression value of said marker gene. This normalized expression value may then be compared with normalized cutoff values, i.e., cutoff values calculated from normalized expression values. In certain embodiments, the control reference gene may be a gene that maintains stable in all samples analyzed in the microarray analysis.

In yet more specific embodiments, the method of the invention may involve comparing the expression values determined for the tested sample with predetermined standard values or cutoff values, or alternatively, with expression values of at least one control sample. As used herein the term “comparing” denotes any examination of the expression level and/or expression values obtained in the samples of the invention as detailed throughout in order to discover similarities or differences between at least two different samples. It should be noted that in some embodiments, comparing according to the present invention encompasses the possibility to use a computer based approach. As described hereinabove, the method of the invention refers to a predetermined cutoff value/s. It should be noted that a “cutoff value”, sometimes referred to simply as “cutoff” herein, is a value that meets the requirements for both high diagnostic sensitivity (true positive rate) and high diagnostic specificity (true negative rate). It should be noted that the terms “sensitivity” and “specificity” are used herein with respect to the ability of one or more markers, to correctly classify a sample as belonging to or fall within the range of a pre-established population associated with intrauterine-transmission of viral pathogens, specifically, CMV, or alternatively, to a pre-established population associated with non-transmission of the viral pathogen to the embryo. “Sensitivity” indicates the performance of the bio-marker of the invention, with respect to correctly classifying samples as belonging to pre-established populations that are likely to transmit intrauterine-transmission of viral pathogens, specifically, CMV, or alternatively, to a pre-established population associated with non-transmission of the viral pathogen to the embryo, when applicable, wherein said bio-marker are consider here as any of the options provided herein.

“Specificity” indicates the performance of the bio-marker of the invention with respect to correctly classifying samples as belonging to or fall within the range of pre-established populations of subjects that are likely to be non-transmitters or unlikely to transmit as will be discussed herein after.

Simply put, “sensitivity” relates to the rate of correct identification of the subjects (samples) as such out of a group of samples, whereas “specificity” relates to the rate of correct identification of transmitting subjects.

In some embodiments, “fall within the range” encompass values that differ from the cutoff value in about 1% to about 50% or more.

It should be emphasized that the nature of the invention is such that the accumulation of further subject data may improve the accuracy of the presently provided cutoff values, which are based on an ROC (Receiver Operating Characteristic) curve generated according to said subject data using analytical software program. The biomarker gene expression values are selected along the ROC curve for optimal combination of prognostic sensitivity and prognostic specificity which are as close to 100 percent as possible, and the resulting values are used as the cutoff values that distinguish between subjects who may transmit the virus or non-transmitting subjects.

Still further, in certain alternative embodiments where a control sample is being used (instead of, or in addition to, pre-determined cutoff values), the normalized expression values of the biomarker genes used by the invention in the test sample are compared to the expression values in the control sample. In certain embodiments, such control sample may be obtained from at least one of a healthy subject (not infected and not pregnant), a healthy pregnant but not infected subject, a non-pregnant subject infected with a viral pathogen, specifically CMV, a pregnant subject known as transmitting at different specific stages of gestation and a subject that is identified as infected non-transmitting subject. In more specific embodiments, predetermined cutoff values may be calculated for a population of subject diagnosed as CMV infected, subjects diagnosed as transmitting infected pregnant subjects, subjects diagnosed as non-transmitting and CMV infected non-pregnant subjects.

It should be appreciated that “Standard” or a “predetermined standard” as used herein, denotes either a single standard value or a plurality of standards with which the level at least one of the biomarker gene expression from the tested sample is compared. The standards may be provided, for example, in the form of discrete numeric values or is calorimetric in the form of a chart with different colors or shadings for different levels of expression; or they may be provided in the form of a comparative curve prepared on the basis of such standards (standard curve).

As note herein before, in some embodiments, the detecting molecules used by the methods, compositions and kits of the invention may comprise nucleic acid-based molecules. As used herein, “nucleic acid molecules” or “nucleic acid sequence” are interchangeable with the term “polynucleotide(s)” and it generally refers to any polyribonucleotide or poly-deoxyribonucleotide, which may be unmodified RNA or DNA or modified RNA or DNA or any combination thereof. “Nucleic acids” include, without limitation, single- and double-stranded nucleic acids. As used herein, the term “nucleic acid(s)” also includes DNAs or RNAs as described above that contain one or more modified bases. Thus, DNAs or RNAs with backbones modified for stability or for other reasons are “nucleic acids”. The term “nucleic acids” as it is used herein embraces such chemically, enzymatically or metabolically modified forms of nucleic acids, as well as the chemical forms of DNA and RNA characteristic of viruses and cells, including for example, simple and complex cells. A “nucleic acid” or “nucleic acid sequence” may also include regions of single- or double-stranded RNA or DNA or any combinations.

As used herein, the term “oligonucleotide” is defined as a molecule comprised of two or more deoxyribonucleotides and/or ribonucleotides, and preferably more than three. Its exact size will depend upon many factors which in turn, depend upon the ultimate function and use of the oligonucleotide. The oligonucleotides may be from about 3 to about 1,000 nucleotides long. Although oligonucleotides of 5 to 100 nucleotides are useful in the invention, preferred oligonucleotides range from about 5 to about 15 bases in length, from about 5 to about 20 bases in length, from about 5 to about 25 bases in length, from about 5 to about 30 bases in length, from about 5 to about 40 bases in length or from about 5 to about 50 bases in length. More specifically, the detecting oligonucleotides molecule used by the composition of the invention may comprise any one of 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 bases in length. It should be further noted that the term “oligonucleotide” refers to a single stranded or double stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions which function similarly.

As indicated throughout, in certain embodiments when the detecting molecules used are nucleic acid based molecules, specifically, oligonucleotides. It should be noted that the oligonucleotides used in here specifically hybridize to nucleic acid sequences of the biomarker genes of the invention. Optionally, where also the expression of at least one of the biomarker genes is being examined, the method of the invention may use as detecting molecules oligonucleotides that specifically hybridize to a nucleic acid sequence of said at least one of the genes. As used herein, the term “hybridize” refers to a process where two complementary nucleic acid strands anneal to each other under appropriately stringent conditions. Hybridizations are typically and preferably conducted with probe-length nucleic acid molecules, for example, 5-100 nucleotides in length, 5-50, 5-40, 5-30 or 5-20.

As used herein “selective or specific hybridization” in the context of this invention refers to a hybridization which occurs between a polynucleotide encompassed by the invention as detecting molecules, and the specific biomarker gene and/or any control reference gene, wherein the hybridization is such that the polynucleotide binds to the gene or any control reference gene preferentially to any other RNA in the tested sample. In a specific embodiment a polynucleotide which “selectively hybridizes” is one which hybridizes with a selectivity of greater than 60 percent, greater than 70 percent, greater than 80 percent, greater than 90 percent and most preferably on 100 percent (i.e. cross hybridization with other RNA species preferably occurs at less than 40 percent, less than 30 percent, less than 20 percent, less than 10 percent). As would be understood to a person skilled in the art, a detecting polynucleotide which “selectively hybridizes” to the biomarker genes or any control reference gene can be designed taking into account the length and composition.

The measuring of the expression of any one of the biomarker genes and any control reference gene or any combination thereof can be done by using those polynucleotides as detecting molecules, which are specific and/or selective for the biomarker genes of the invention to quantitate the expression of said biomarker genes or any control reference gene. In a specific embodiment of the invention, the polynucleotides which are specific and/or selective for said genes may be probes or a pair of primers. It should be further appreciated that the methods, as well as the compositions and kits of the invention may comprise, as an oligonucleotide-based detection molecule, both primers and probes.

The term, “primer”, as used herein refers to an oligonucleotide, whether occurring naturally as in a purified restriction digest, or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be single-stranded or double-stranded and must be sufficiently long to prime the synthesis of the desired extension product in the presence of the inducing agent. The exact length of the primer will depend upon many factors, including temperature, source of primer and the method used. For example, for diagnostic applications, depending on the complexity of the target sequence, the oligonucleotide primer typically contains 10-30 or more nucleotides, although it may contain fewer nucleotides. More specifically, the primer used by the methods, as well as the compositions and kits of the invention may comprise 10, 11, 12, 13, 14, 15, 16, 17, 18, 19, 20, 21, 22, 23, 24, 25, 26, 27, 28, 29 or 30 nucleotides or more. In certain embodiments, such primers may comprise 30, 40, 50, 60, 70, 80, 90, 100 nucleotides or more. In specific embodiments, the primers used by the method of the invention may have a stem and loop structure. The factors involved in determining the appropriate length of primer are known to one of ordinary skill in the art and information regarding them is readily available.

As used herein, the term “probe” means oligonucleotides and analogs thereof and refers to a range of chemical species that recognize polynucleotide target sequences through hydrogen bonding interactions with the nucleotide bases of the target sequences. The probe or the target sequences may be single- or double-stranded RNA or single- or double-stranded DNA or a combination of DNA and RNA bases. A probe is at least 5 or preferably, 8 nucleotides in length. A probe may be 5, 6, 7, 8, 9, 10, 11, 12, 13, 14, 15, 16, 17, 18, 19, 20, 21, 22, 23, 24, 25, 26, 27, 28, 29 and up to 30 nucleotides in length as long as it is less than the full length of the target marker gene. Probes can include oligonucleotides modified so as to have a tag which is detectable by fluorescence, chemiluminescence and the like. The probe can also be modified so as to have both a detectable tag and a quencher molecule, for example TaqMan® and Molecular Beacon® probes, that will be described in detail below.

The oligonucleotides and analogs thereof may be RNA or DNA, or analogs of RNA or DNA, commonly referred to as antisense oligomers or antisense oligonucleotides. Such RNA or DNA analogs comprise, but are not limited to, 2-′O-alkyl sugar modifications, methylphosphonate, phosphorothiate, phosphorodithioate, formacetal, 3-thioformacetal, sulfone, sulfamate, and nitroxide backbone modifications, and analogs, for example, LNA analogs, wherein the base moieties have been modified. In addition, analogs of oligomers may be polymers in which the sugar moiety has been modified or replaced by another suitable moiety, resulting in polymers which include, but are not limited to, morpholino analogs and peptide nucleic acid (PNA) analogs. Probes may also be mixtures of any of the oligonucleotide analog types together or in combination with native DNA or RNA. At the same time, the oligonucleotides and analogs thereof may be used alone or in combination with one or more additional oligonucleotides or analogs thereof.

Thus, according to one embodiment, such oligonucleotides are any one or at least one of a pair of primers or nucleotide probes, and wherein the level of expression of at least one of the biomarker genes is determined using a nucleic acid amplification assay selected from the group consisting of: a Real-Time PCR, micro array, PCR, in situ hybridization and comparative genomic hybridization.

The term “amplification assay”, with respect to nucleic acid sequences, refers to methods that increase the representation of a population of nucleic acid sequences in a sample. Nucleic acid amplification methods, such as PCR, isothermal methods, rolling circle methods, etc., are well known to the skilled artisan. More specifically, as used herein, the term “amplified”, when applied to a nucleic acid sequence, refers to a process whereby one or more copies of a particular nucleic acid sequence is generated from a template nucleic acid, preferably by the method of polymerase chain reaction. “Polymerase chain reaction” or “PCR” refers to an in vitro method for amplifying a specific nucleic acid template sequence. The PCR reaction involves a repetitive series of temperature cycles and is typically performed in a volume of 50-100 microliter. The reaction mix comprises dNTPs (each of the four deoxynucleotides dATP, dCTP, dGTP, and dTTP), primers, buffers, DNA polymerase, and nucleic acid template. The PCR reaction comprises providing a set of polynucleotide primers wherein a first primer contains a sequence complementary to a region in one strand of the nucleic acid template sequence and primes the synthesis of a complementary DNA strand, and a second primer contains a sequence complementary to a region in a second strand of the target nucleic acid sequence and primes the synthesis of a complementary DNA strand, and amplifying the nucleic acid template sequence employing a nucleic acid polymerase as a template-dependent polymerizing agent under conditions which are permissive for PCR cycling steps of (i) annealing of primers required for amplification to a target nucleic acid sequence contained within the template sequence, (ii) extending the primers wherein the nucleic acid polymerase synthesizes a primer extension product. “A set of polynucleotide primers”, “a set of PCR primers” or “pair of primers” can comprise two, three, four or more primers.

Real time nucleic acid amplification and detection methods are efficient for sequence identification and quantification of a target since no pre-hybridization amplification is required. Amplification and hybridization are combined in a single step and can be performed in a fully automated, large-scale, closed-tube format.

Methods that use hybridization-triggered fluorescent probes for real time PCR are based either on a quench-release fluorescence of a probe digested by DNA Polymerase (e.g., methods using TaqMan®, MGB-TaqMan®), or on a hybridization-triggered fluorescence of intact probes (e.g., molecular beacons, and linear probes). In general, the probes are designed to hybridize to an internal region of a PCR product during annealing stage (also referred to as amplicon). For those methods utilizing TaqMan® and MGB-TaqMan® the 5′-exonuclease activity of the approaching DNA Polymerase cleaves a probe between a fluorophore and a quencher, releasing fluorescence.

Thus, a “real time PCR” or “RT-PCT” assay provides dynamic fluorescence detection of amplified genes or any control reference gene produced in a PCR amplification reaction. During PCR, the amplified products created using suitable primers hybridize to probe nucleic acids (TaqMan® probe, for example), which may be labeled according to some embodiments with both a reporter dye and a quencher dye. When these two dyes are in close proximity, i.e. both are present in an intact probe oligonucleotide, the fluorescence of the reporter dye is suppressed. However, a polymerase, such as AmpliTaq Gold™, having 5′-3′ nuclease activity can be provided in the PCR reaction. This enzyme cleaves the fluorogenic probe if it is bound specifically to the target nucleic acid sequences between the priming sites. The reporter dye and quencher dye are separated upon cleavage, permitting fluorescent detection of the reporter dye. Upon excitation by a laser provided, e.g., by a sequencing apparatus, the fluorescent signal produced by the reporter dye is detected and/or quantified. The increase in fluorescence is a direct consequence of amplification of target nucleic acids during PCR. The method and hybridization assays using self-quenching fluorescence probes with and/or without internal controls for detection of nucleic acid application products are known in the art, for example, U.S. Pat. Nos. 6,258,569; 6,030,787; 5,952,202; 5,876,930; 5,866,336; 5,736,333; 5,723,591; 5,691,146; and 5,538,848. More particularly, QRT-PCR or “qPCR” (Quantitative RT-PCR), which is quantitative in nature, can also be performed to provide a quantitative measure of gene expression levels. In QRT-PCR reverse transcription and PCR can be performed in two steps, or reverse transcription combined with PCR can be performed. One of these techniques, for which there are commercially available kits such as TaqMan® (Perkin Elmer, Foster City, Calif.), is performed with a transcript-specific antisense probe. This probe is specific for the PCR product (e.g. a nucleic acid fragment derived from a gene, or in this case, from a pre-miRNA) and is prepared with a quencher and fluorescent reporter probe attached to the 5′ end of the oligonucleotide. Different fluorescent markers are attached to different reporters, allowing for measurement of at least two products in one reaction.

When Taq DNA polymerase is activated, it cleaves off the fluorescent reporters of the probe bound to the template by virtue of its 5-to-3′ exonuclease activity. In the absence of the quenchers, the reporters now fluoresce. The color change in the reporters is proportional to the amount of each specific product and is measured by a fluorometer; therefore, the amount of each color is measured and the PCR product is quantified. The PCR reactions can be performed in any solid support, for example, slides, microplates, 96 well plates, 384 well plates and the like so that samples derived from many individuals are processed and measured simultaneously. The TaqMan® system has the additional advantage of not requiring gel electrophoresis and allows for quantification when used with a standard curve.

A second technique useful for detecting PCR products quantitatively without is to use an intercalating dye such as the commercially available QuantiTect SYBR Green PCR (Qiagen, Valencia Calif.). RT-PCR is performed using SYBR green as a fluorescent label which is incorporated into the PCR product during the PCR stage and produces fluorescence proportional to the amount of PCR product.

Both TaqMan® and QuantiTect SYBR systems can be used subsequent to reverse transcription of RNA. Reverse transcription can either be performed in the same reaction mixture as the PCR step (one-step protocol) or reverse transcription can be performed first prior to amplification utilizing PCR (two-step protocol).

Additionally, other known systems to quantitatively measure mRNA expression products include Molecular Beacons® which uses a probe having a fluorescent molecule and a quencher molecule, the probe capable of forming a hairpin structure such that when in the hairpin form, the fluorescence molecule is quenched, and when hybridized, the fluorescence increases giving a quantitative measurement of gene expression.

According to this embodiment, the detecting molecule may be in the form of probe corresponding and thereby hybridizing to any region or part of the biomarker genes or any control reference gene. More particularly, it is important to choose regions which will permit hybridization to the target nucleic acids. Factors such as the Tm of the oligonucleotide, the percent GC content, the degree of secondary structure and the length of nucleic acid are important factors.

It should be further noted that a standard Northern blot assay can also be used to ascertain an RNA transcript size and the relative amounts of the biomarker genes or any control gene product, in accordance with conventional Northern hybridization techniques known to those persons of ordinary skill in the art.

The invention further contemplates the use of amino acid based molecules such as proteins or polypeptides as detecting molecules disclosed herein and would be known by a person skilled in the art to measure the protein products of the marker genes of the invention. Techniques known to persons skilled in the art (for example, techniques such as Western Blotting, Immunoprecipitation, ELISAs, protein microarray analysis, Flow cytometry and the like) can then be used to measure the level of protein products corresponding to the biomarker of the invention. As would be understood to a person skilled in the art, the measure of the level of expression of the protein products of the biomarker of the invention requires a protein, which specifically and/or selectively binds to the biomarker genes of the invention.

As indicated above, the detecting molecules of the invention may be amino acid based molecules that may be referred to as protein/s or polypeptide/s. As used herein, the terms “protein” and “polypeptide” are used interchangeably to refer to a chain of amino acids linked together by peptide bonds. In a specific embodiment, a protein is composed of less than 200, less than 175, less than 150, less than 125, less than 100, less than 50, less than 45, less than 40, less than 35, less than 30, less than 25, less than 20, less than 15, less than 10, or less than 5 amino acids linked together by peptide bonds. In another embodiment, a protein is composed of at least 200, at least 250, at least 300, at least 350, at least 400, at least 450, at least 500 or more amino acids linked together by peptide bonds. It should be noted that peptide bond as described herein is a covalent amid bond formed between two amino acid residues.

In specific embodiments, the detecting amino acid molecules are isolated antibodies, with specific binding selectively to the proteins encoded by the biomarker genes as detailed above. Using these antibodies, the level of expression of proteins encoded by the genes may be determined using an immunoassay which is selected from the group consisting of FACS, a Western blot, an ELISA, a RIA, a slot blot, a dot blot, immunohistochemical assay and a radio-imaging assay.

In yet other specific embodiments, the method of the invention may use any sample. In more specific embodiment, such sample may be any one of peripheral blood mononuclear cells, amniotic fluid, and biopsies of organs or tissues.

It should be noted that any of the detecting molecules used by the methods, compositions and kits of the invention are isolated and purified. Still further, it must be understood that any of the detecting molecules (for example, primers and/or probes) or reagents used by the compositions, kits, arrays and in any step of the methods of the invention are non-naturally occurring products or compounds, As such, none of the detecting molecules of the invention are directed to naturally occurring compounds or products.

Still further, such detecting molecule may be at least one of a pair of primers or nucleotide probes.

In one embodiment, the polynucleotide-based detection molecules of the invention may be in the form of nucleic acid probes which can be spotted onto an array to measure RNA from the sample of a subject to be diagnosed.

As defined herein, a “nucleic acid array” refers to a plurality of nucleic acids (or “nucleic acid members”), optionally attached to a support where each of the nucleic acid members is attached to a support in a unique pre-selected and defined region. These nucleic acid sequences are used herein as detecting nucleic acid molecules. In one embodiment, the nucleic acid member attached to the surface of the support is DNA. In a preferred embodiment, the nucleic acid member attached to the surface of the support is either cDNA or oligonucleotides. In another embodiment, the nucleic acid member attached to the surface of the support is cDNA synthesized by polymerase chain reaction (PCR). In another embodiment, a “nucleic acid array” refers to a plurality of unique nucleic acid detecting molecules attached to nitrocellulose or other membranes used in Southern and/or Northern blotting techniques. For oligonucleotide-based arrays, the selection of oligonucleotides corresponding to the gene of interest which are useful as probes is well understood in the art.

As indicated above, assay based on micro array or RT-PCR may involve attaching or spotting of the probes in a solid support. As used herein, the terms “attaching” and “spotting” refer to a process of depositing a nucleic acid onto a substrate to form a nucleic acid array such that the nucleic acid is stably bound to the substrate via covalent bonds, hydrogen bonds or ionic interactions.

As used herein, “stably associated” or “stably bound” refers to a nucleic acid that is stably bound to a solid substrate to form an array via covalent bonds, hydrogen bonds or ionic interactions such that the nucleic acid retains its unique pre-selected position relative to all other nucleic acids that are stably associated with an array, or to all other pre-selected regions on the solid substrate under conditions in which an array is typically analyzed (i.e., during one or more steps of hybridization, washes, and/or scanning, etc.).

As used herein, “substrate” or “support” or “solid support”, when referring to an array, refers to a material having a rigid or semi-rigid surface. The support may be biological, non-biological, organic, inorganic, or a combination of any of these, existing as particles, strands, precipitates, gels, sheets, tubing, spheres, beads, containers, capillaries, pads, slices, films, plates, slides, chips, etc. Often, the substrate is a silicon or glass surface, (poly)tetrafluoroethylene, (poly) vinylidendifmoride, polystyrene, polycarbonate, a charged membrane, such as nylon or nitrocellulose, or combinations thereof. Preferably, at least one surface of the substrate will be substantially flat. The support may optionally contain reactive groups, including, but not limited to, carboxyl, amino, hydroxyl, thiol, and the like. In one embodiment, the support may be optically transparent. As noted above, the solid support may include polymers, such as polystyrene, agarose, sepharose, cellulose, glass, glass beads and magnetizable particles of cellulose or other polymers. The solid-support can be in the form of large or small beads, chips or particles, tubes, plates, or other forms.

The inventors consider the kit of the invention in compartmental form. It should be therefore noted that the detecting molecules used for detecting the expression levels of the biomarker genes may be provided in a kit attached to an array. As defined herein, a “detecting molecule array” refers to a plurality of detection molecules that may be nucleic acids based or protein based detecting molecules (specifically, probes, primers and antibodies), optionally attached to a support where each of the detecting molecules is attached to a support in a unique pre-selected and defined region.

For example, an array may contain different detecting molecules, such as specific antibodies or primers. As indicated herein before, in case a combined detection of the biomarker genes expression level, the different detecting molecules for each target may be spatially arranged in a predetermined and separated location in an array. For example, an array may be a plurality of vessels (test tubes), plates, micro-wells in a micro-plate, each containing different detecting molecules, specifically, probes, primers and antibodies, against polypeptides encoded by the marker genes used by the invention. An array may also be any solid support holding in distinct regions (dots, lines, columns) different and known, predetermined detecting molecules.

As used herein, “solid support” is defined as any surface to which molecules may be attached through either covalent or non-covalent bonds. Thus, useful solid supports include solid and semi-solid matrixes, such as aero gels and hydro gels, resins, beads, biochips (including thin film coated biochips), micro fluidic chip, a silicon chip, multi-well plates (also referred to as microtiter plates or microplates), membranes, filters, conducting and no conducting metals, glass (including microscope slides) and magnetic supports. More specific examples of useful solid supports include silica gels, polymeric membranes, particles, derivative plastic films, glass beads, cotton, plastic beads, alumina gels, polysaccharides such as Sepharose, nylon, latex bead, magnetic bead, paramagnetic bead, super paramagnetic bead, starch and the like. This also includes, but is not limited to, microsphere particles such as Lumavidin™ Or LS-beads, magnetic beads, charged paper, Langmuir-Blodgett films, functionalized glass, germanium, silicon, PTFE, polystyrene, gallium arsenide, gold, and silver. Any other material known in the art that is capable of having functional groups such as amino, carboxyl, thiol or hydroxyl incorporated on its surface, is also contemplated. This includes surfaces with any topology, including, but not limited to, spherical surfaces and grooved surfaces.

It should be further appreciated that any of the reagents, substances or ingredients included in any of the methods and kits of the invention may be provided as reagents embedded, linked, connected, attached, placed or fused to any of the solid support materials described above.

According to other embodiments, the kit of the invention may be suitable for examining samples such as peripheral blood mononuclear cells, amniotic fluid and biopsies of organs or tissues.

It should be appreciated that the method of the invention may be also applicable as a predictive tool to screen subjects, specifically, human subjects prior to pregnancy, thereby identifying subjects, specifically human subjects or populations at risk that may be vulnerable to and thereby may exhibit intrauterine-transmission of viral pathogens, specifically CMV.

It should be further appreciated that in addition to the diagnostic and predictive methods, compositions and kits, the invention further provides in yet some embodiments thereof prophylactic methods and tools for preventing and reducing the incidence or risk of pathologic injuries caused by intrauterine-transmission of viral pathogens, specifically of CMV. In yet more specific embodiments, such pathologic conditions, clinical manifestations or injuries may include but are not limited to intrauterine growth retardation, purpura, jaundice, hepatosplenomegaly, microencephaly, hearing and vision impairment, thrombocytopenia as well as any of the injuries disclosed herein before.

All scientific and technical terms used herein have meanings commonly used in the art unless otherwise specified. The definitions provided herein are to facilitate understanding of certain terms used frequently herein and are not meant to limit the scope of the present disclosure.

As used herein the term “about” refers to ±10% The terms “comprises”, “comprising”, “includes”, “including”, “having” and their conjugates mean “including but not limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%. The terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “about” as used herein indicates values that may deviate up to 1 percent, more specifically 5 percent, more specifically 10 percent, more specifically 15 percent, and in some cases up to 20 percent higher or lower than the value referred to, the deviation range including integer values, and, if applicable, non-integer values as well, constituting a continuous range.

It must be noted that, as used in this specification and the appended claims, the singular forms “a”, “an” and “the” include plural referents unless the content clearly dictates otherwise.

### EXAMPLES

### Experimental Procedures

a. Study Sample

Participants attending the retrospective examination of Shaare Zedek Medical Center, Jerusalem, Israel were eligible for inclusion in this study. The institutional review board of Shaare Zedek Medical Center approved the protocol and participants provided written informed-consent. All participants provided a medical history and underwent a physical examination and laboratory assessment of immune for primary hCMV infection and viral load during the first and second trimesters of pregnancy.

Blood samples were sequentially collected from pregnant women diagnosed with primary CMV infection. Table 1 summarizes the data on 18 hCMV infected pregnant woman participating in this study.

TABLE 1
List and the data of the 18 hcmv infected woman participating in the study.

| | | | | | Gestational | Gestational | |
|---|---|---|---|---|---|---|---|
| | | | | CMV | age of | age at | |
| | | | | Copies | CMV | blood | Avidity |
| Patient | | Pregnancy | | in ml | Infection | sampling | Index |
| code | Age | No. | Group | blood | (week) | (week) | (%) |
| 07GR | 30 | 2 | T | 307 | 24.0 | 29 | 12.0% |
| 19TA | 29 | 2 | T | 173087 | 14.0 | 20 | 15.0% |
| 22SH | 26 | 1 | T | 2456 | 10.0 | 17 | 8.5% |
| 37LH | 32 | 2 | T | 837 | 18.0 | 22 | 14.6% |
| 43VE | 27 | 2 | T | 207 | 20.0 | 24 | 7.6% |
| 06RB | 37 | 8 | NT | 231 | 6.0 | 14 | 18.0% |
| 08PA | 28 | 3 | NT | 2926 | 6.0 | 16 | 22.6% |
| 10GC | 23 | 2 | NT | 2643 | 11.0 | 17 | 12.0% |
| 13SR | 30 | 3 | NT | 952 | 11.5 | 19 | 8.2% |
| 16YM | 22 | 2 | NT | 2231 | 20.0 | 28 | Neg. |
| 17SA | 31 | 1 | NT | 48547 | 2.5 | 8 | 8.1% |
| 67MA | 27 | 6 | NT | 596 | 21.0 | 26 | 11.1% |
| 68KA | 36 | 5 | NT | 1573 | 7.0 | 13 | 11.5% |
| 28RR | 25 | 3 | NT | 1865 | 14.0 | 18 | 15.0% |
| 32HL | 33 | 6 | NT | 872 | 5.0 | 13 | 20.0% |
| 35PC | 29 | 13 | NT | 315 | 6.0 | 9 | 2.3% |
| 42GM | 25 | 2 | NT | 633 | 10.0 | 16 | 17.4% |
| 75TT | 31 | 3 | NT | 408 | 4 | 12 | 10% |

### Timing of Primary CMV Infection and Intrauterine Transmission:

Primary infection timing was determined by the time point of seroconversion and/or analysis of the increment of IgG avidity and/or by clinical symptoms²⁵. Intrauterine CMV transmission was diagnosed by detection of viral DNA by shell vial culture and/or real-time PCR, either in amniotic fluid or in the newborn's urine.

b. Biomarker Selection and Measurement Outcomes.

Five microarray transcriptome datasets consist of in vitro uninfected and hCMV infected PBMC at 4-48 hrs post infection (hpi). The expression levels were obtained from publicly available data bases lhttp://www.ncbi.nlm nih.gov/geo/1 using the following Gene Expression Omnibus Accession Nos:

I. Gene Expression Omnibus Accession No. GSE14490; describes analysis of dendritic cells derived from 6 mock infected and matching 6 hCMV infected donors, 6 hpi.
II. Gene Expression Omnibus Accession No. GSE14816; describes analysis of dendritic cells derived from 3 mock infected and matching 3 hCMV infected donors, 24 hpi.
III. Gene Expression Omnibus Accession No. GSE17948; describes analysis of monocytes derived from 4 mock infected and matching 4 hCMV infected donors, 24 hpi.
IV. Gene Expression Omnibus Accession No. GSE19772; describes analysis of monocytes derived from 2 mock infected and matching 2 hCMV infected donors, 48 hpi.
V. Gene Expression Omnibus Accession No. GSE14408; describes analysis of monocytes cells derived from 6 mock infected and matching 6 hCMV infected donors, 4 hpi.

In addition, Gene Expression Omnibus Accession No. GSE63797 performing deep sequencing of uninfected or infected human foreskin fibroblasts with hCMV (24, 48 and 72 post-infection hour) was used for verification.

### Screening for Biomarkers:

The five datasets (I-V described above) included matching pairs of PBMC in vitro mock treated and hCMV infected 4-48 hpi.

After transforming the data from these databases to the same scale, the five datasets were combined into one data group consisting of mock infected samples followed by their matching infected ones. Conducting a volcano analysis (using Matlab Version 13), a list of genes satisfying the conditions of >2 fold changes and p-value lower than 0.05 was generated (FIG. 1A).

Selecting genes with False discovery rate (FDR) restriction of less than 0.05 from combined broad list in STRING (Search tool for the retrieval of interacting genes/proteins-Ver. 9.1) database clearly indicates the major role of the IFN pathway genes in response 4-48 h post CMV infection in PBMC taken from healthy donors (FIG. 1B). In particular the key role of the IFIT, OAS, IFI, and Ubiquitin families was observed.

A similar set of genes was also indicated by a volcano analysis on a deep sequencing dataset GSE63797 comparing uninfected to infected human foreskin fibroblasts with HCMV 24 hpi (not shown).

Thus, it was decided to test representatives of these gene families in CMV primary infected pregnant woman. Indeed, 3-4 of these tested genes were sufficient to predict the transmission of the virus to the fetus.

c. Laboratory Tests:

Primary CMV infection was determined by CMV-specific IgG seroconversion, or the presence of low avidity IgG antibodies or CMV-specific IgM with no previous IgG antibodies or the presence of CMV or viral products in the blood.

Viral Load-CMV viremia was performed by a real-time PCR reaction. 200 copy number/ml blood was the limit of detection of this assay.

### qRT PCR:

Total RNA was purified from the PBMC obtained from pregnant women by using RNAqueous® Kit (AM 1912 Life Technology) and stored at −70° C. until use. The RNA preparations were converted into cDNA by using High Capacity cDNA Reverse Transcription Kit (AB-4374966 Life Technology) according to the company instructions. For gene expression analysis, the QuantStudio 12K Flex PCR system Life Technology) was used, where the reaction mix contained: TaqMan Gene Expression Master Mix (2×) 5 μl, and 12 ng CDNA in 0.4 μl H₂O and H₂O 4.1 μl.

In parallel, the ‘no template control’ (NTC) contained the same components without cDNA. A volume of 0.5 μl of TaqMan Gene Expression Assay (20×) enzyme was added to all samples, which were pre-mixed with the appropriate primers, The PCR reactions were carried out under the following conditions: 2 min at 50° C., 10 min at 95° C. for denaturation and 40 cycles of 15 sec in 95° C. followed by 60° C. for 1 min. We have evaluated the suitability of GAPDH, B2M and RPLPO for use as normalizing genes. GAPDH was discarded since is expression levels were correlate with the IFN genes. B2M was selected as the most suitable candidate.

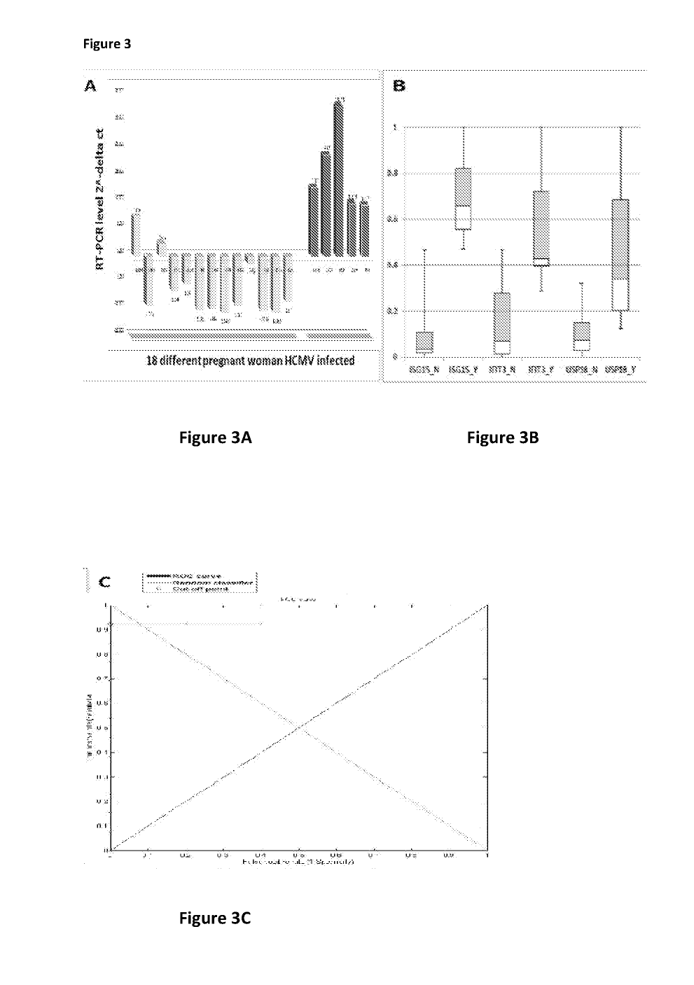

d. Statistical Analysis—ROC Using the three genes, IFIT3, IS15 and USP18 with the lowest ranking P-values, a ROC curve analysis was performed (FIG. 3C) with Matlab (Version R2013a). The area under the curve was 0.9846 with a standard error of 0.02716 and 95& C.I between 0.93137 and 1. Table at cut-off point

| | | |
|---|---|---|
| Actual Results | Prediction NT | Prediction T |

| | | | |
|---|---|---|---|
| Non Trans | 3 | 12 | 1 |
| Trans | | 0 | 5 |

Sensitivity (1-probability that test is a transmitter on non-transmitter subject): 92.3% 95% confidence interval: 77.8%-100.0%
False negative proportion: 7.7%
Specificity (1-probability that test is a non-transmitter on a transmitter subject): 100.0% 95% confidence interval: 100.0%-100.0%
False positive proportion: 0.0%

### F-measure: 96.0%

### Accuracy or Potency: 94.4%

### Mis-classification Rate: 5.6%

### Example 1

Activation of Cellular Genes by hCMV Infection

The effect of hCMV infection on cellular genes expression in PBMC was described in five available published array-datasets, each one consists of several patients. Uninfected cells were used as references for defining the hCMV-dependent gene expression changes.

A volcano plot derived from a group that integrated all the five datasets showed the genes that were overexpressed as a result of hCMV infection (FIG. 1A). The evaluation of the upregulated genes derived from the volcano analysis in String analysis (FIG. 1B) showed the key role of the IFIT, OAS, IFI, and Ubiquitin members.

In order to predict the response of each woman to hCMV infection, the expression of the ISG15 gene was first compared in all the patients. FIGS. 1C-1G show that most (17 out of 21) women respond to the viral infection by augmentation of ISG15 gene expression, while in cells derived from 4 patients (19%), the level of ISG15 gene expression was mildly increased.

These results were further corroborated by assessing the response of the 21 patients to the IFIT, OAS, IFI, and Ubiquitin IFN-signaling genes that were parallel to the ISG15 dynamics for each case. These data, clearly showed that representatives of these families of IFN-signaling can serve as a reliable set for selection bio-markers for differentiation between transmitters and non-transmitters individuals. Namely, women who are considered transmitters (transmit hCMV) and those who are considered as non-transmitters (will not transmit hCMV).

### Example 2

### Mathematical Model Elaborating Equilibrium Between Virus Replication and IFN-Stimulated Genes Expression

It is believed that viral infected cells sense pathogen-associated molecular patterns (PAMPs) and initiate an innate response i.e. a counter campaign resulting in reducing the amount of the replicating virus.

Rescuing the cellular anti-viral response, hCMV expresses large number of genes committed to neutralize the innate immune genes, such as IE1-p72 and IE-86 (19)

The mathematical model simulates and predicts the time course of the interaction between the replication of viruses within a single cell and their counter fighting cell proteins. The model suggests continues repeat of two consecutive cycles process; a virus action cycle followed by cell's reaction cycle.

The factor K represents the constant virus replication in each cycle. This factor equals the ability of a virus to hijack cellular mechanisms and replicate at a specific pace. The factor M represents the portion (between 0 to 1) of the existing population of (intracellular) viruses eliminated by the cell defense mechanisms. As consequence the equilibrium point can be determined M=1−1/K. Finally, N is the current cycle count of the virus versus cell battle. Thus at the beginning of cycle N there are X(N−1) viruses in the cell. At the end of the cycle this number can be calculated by the following equation as:

X(N)=X(N−1)*K.

This model assumes that the fighting proteins retaliate in the following cycle. The amount of the cells fighting proteins Y during cycle N remains unchanged:

Y(N)=Y(N−1).

In the following cycle N+1, the fighting proteins eliminate a portion M of the existing population of viruses thus:

X(N+1)=X(N)−M*X(N)

therefore the amount of fighting proteins needed for such elimination can be expressed as

Y(N+1)=M*X(N).

The repeating process in its discrete form can be written as follows:

X(N)=X(N−1)*K

Y(N)=Y(N−1)

X(N+1)=X(N)−M*X(N)

whereas: Y(N+1)=M*X(N).

K value of different viruses can be obtained from their doubling time provided by means such as cell impedance measurements.

The doubling times for WNV and SLEV were estimated to be 4.0 hr and 13.5 hr²¹, respectively, indicating that WNV can proliferate at a rate nearly three-times faster than SLEV. Similarly HCV was assumed to have a doubling time of 6 hours and HIV 10 hours. Thus in 12 hr HCV in each cell will have greater than 4 copies so we can assume K=5.

In the case of hCMV multiply in allogeneic hematopoietic stem cell transplant (HSCT) patients, Cromer and her co-workers²⁰ estimated the doubling time of hCMV 0.96/day, or the number of particles multiplied by a factor of 1.92 per 24 hr (K=1.92).

Thus assuming this K in pregnancy for the woman to clear the virus as soon as possible, such a woman M value should be greater than 1−1/1.92=0.4792 preferably as close to 1 as possible.

For a K=2 of hCMV, the model suggests that non transmitters should have an M value greater than 0.5 (M=1−1/K=0.5) and less than 0.5 for the transmitters.

Accordingly, by calculating M from RT PCR of ISG15 gene expression level, the non-transmitters group was found to have M values close to 0 and the transmitters group was found to have M values close to 1.

calculating the M value of the sample (Kₛₐₘₚ) may be performed by the steps of: (a) obtaining the expression value Exₛₐₘₚ of said sample as determined by the method of the invention; (b) providing a standard curve of expression values of hCMV infected subjects; (c) obtaining a maximal expression value Exₘₐₓ and a minimal expression value Exₘᵢₙ from said standard curve of (b); and (d) calculating the Mₛₐₘₚ value of said sample, wherein Mₛₐₘₚ=1−[(Exₛₐₘₚ−Exₘᵢₙ)/(Exₘₐₓ−Exₘᵢₙ)].

Similarly, the values of Kₐₘₚ may be calculated using the equivalent equation:

Mₛₐₘₚ=(Exₘₐₓ−Exₛₐₘₚ)/(Exₘₐₓ−Exₘᵢₙ₎].

With the doubling time range of 1.92 hr to 13.5 hr, the model suggests the cells dedicated fighting genes should up regulate their expression 4-8 fold in 12 hours within virus detection. This again agrees with the data obtained from the microarray datasets we used.

The M factor is personal and varies between people and it can be considered to be the main factor that determines the outcome of the battle between the cell and the virus by sustaining a gap from the equilibrium at M=1−1/K.

The inventors demonstrated that the gap M to equilibrium 1−1/K ratio can explain the virus load variability measured on the one hand and cell's fighting genes expression dynamics on the other hand. The cells response to meet the increased virus replication, according to this model, is by attempting to increase the expression of the responding IFN genes. The consequence is that cells can win the viral replication machinery by fast up regulation of the IFN fighting genes, as soon as possible to the virus entry.

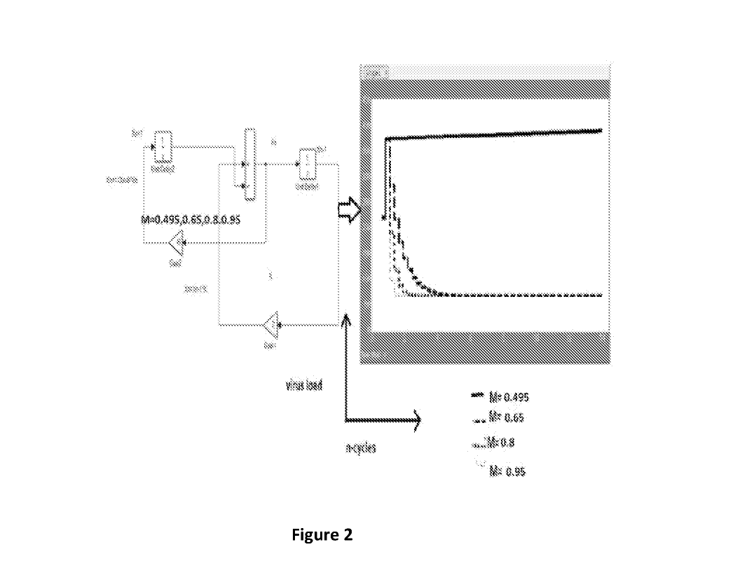

FIG. 2 shows a simulation of the model for reacting to hCMV with 4 different M values.

When M=0.495 the virus replication out paces the cell fighting gene resulting in a measured high gene expressions levels and a transmission to the fetus. When M=0.65, 0.8 or 0.95 the cells fighting genes prevail the virus replication and block the transmission to the fetus.

The model also predicts the following virus load dosing relationship

1) The decrease of virus load in all treatment composed of an initial steep decline followed by slower and longer decline.
2) Increasing dosing should result in a sharper and faster decline of virus load.
3) The difference in virus load decline in a treatment is depended solely on a person's ability to quickly up regulate his IFN fighting genes.

### Example 3

Expression of IFN Signaling Genes in hCMV Infected Pregnant Women

Primary hCMV infection of a pregnant woman confers the highest risk of congenital infection and disease. Approximately 40 percent of hCMV infected women are expected to transmit the virus to their fetus. The inventors were interested to reveal whether the level of small set of bio-markers expression can be used for identifying women in risk to confer intrauterine-virus-transmission.

The inventors extracted total cellular RNA from stored frozen PMBC collected from 27 consent pregnant women: 18 with hCMV load of at least 200 copies/ml blood. As the model requires triggering by the virus, samples with undetected viral load were excluded. Analyses of the selected bio-markers expression by qRT-PCR strongly suggest that the hCMV infected pregnant women are divided into two groups; twelve pregnant women express low levels, while 6 others express high levels of most of the bio-markers.

FIG. 3A shows the sum of the expression of 3 selected gene (ISG15, IFIT3 and USP18) in 18 hCMV infected pregnant women, emphasizing the differences between the two groups of women.

To further substantiate the results, the inventors drew a Box Plot showing the expression level of 3 selected genes (Table 2) of the two populations of women (FIG. 3B).

The ROC curve calculated for the Zscored normalized sum of the expression of the three genes in the two groups is shown in FIG. 3C. These results (FIG. 3A-C) clearly indicate that the 18 women can be divided into two groups distinguished by the expression levels of the IFN-signaling genes.

More specifically, FIG. 3A shows that the women can be divided into two separate group: 5 transmitters with high genes expression level and 12 non transmitters with low genes expression level. The expression level of 3 genes (FIG. 3A) suggested that woman 16YM is a marginal case. This could lead to undesired clinical conclusions.

The results of prediction are:

### Accuracy=94.4%

### Sensitivity=92.3%

### Specificity=100%

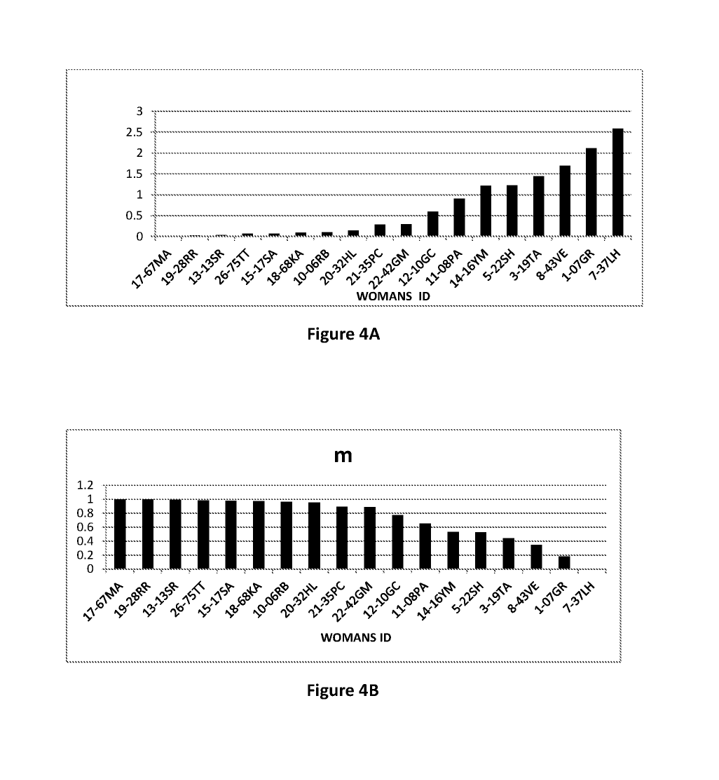

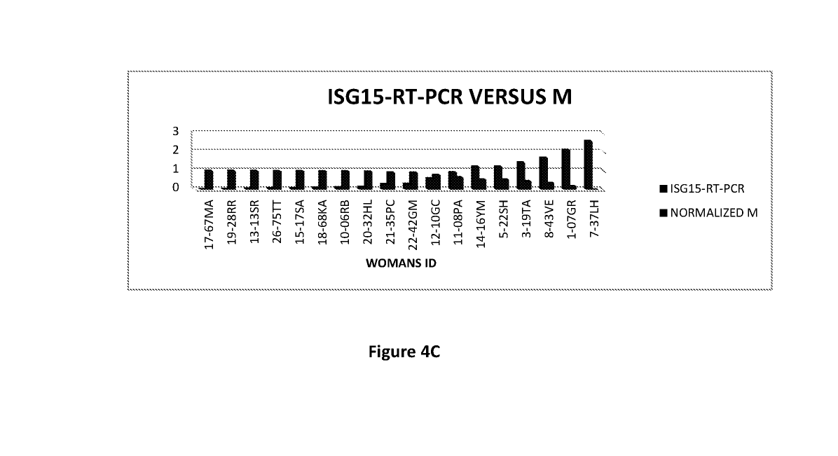

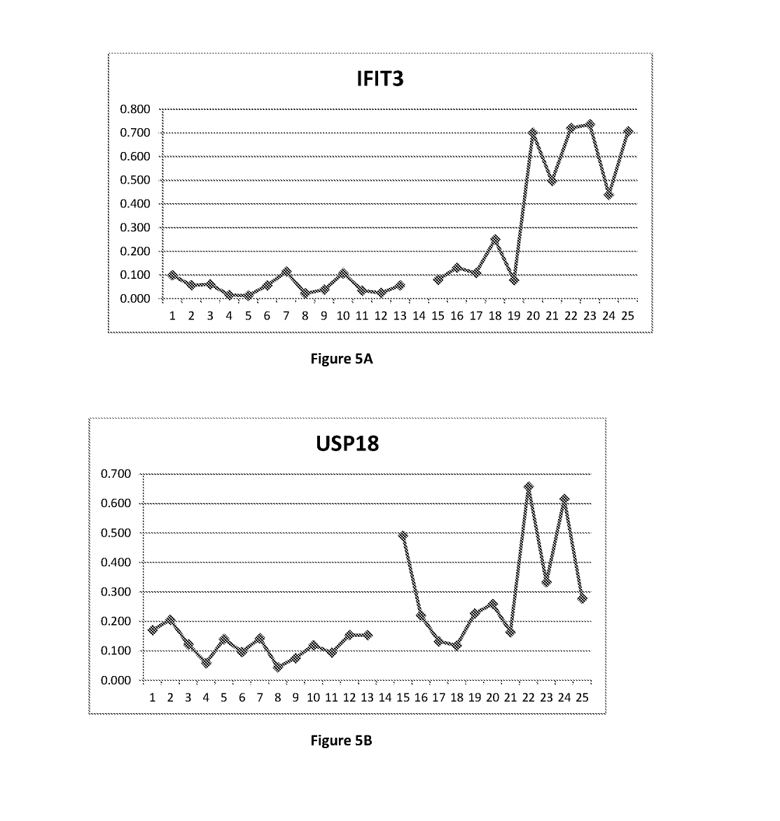

FIG. 4A-C demonstrates the calculation of Mₛₐₘₚ for all 18 hCMV infected patients using the levels expression of ISG15 gene, as measured by RT-PCR. The expression levels are indicated in Table 2 below. Thus, using the equation Mₛₐₘₚ=(Exₘₐₓ−Exₛₐₘₚ)/(Exₘₐₓ−Exₘᵢₙ)], one can calculate the M levels of each examined subject. For example, as shown in FIG. 4A and Table 2, the minimal expression of ISG15, is of patient 17-67MA (Exₘᵢₙ=0.019), the maximal expression level is of patient 7-37LH (Exₘₐₓ=2.581). Thus, the M of patient 14-16YM (Exₛₐₘₚ=1.217) is:

Mₛₐₘₚ=(EXₘₐₓ−Exₛₐₘₚ)/(Exₘₐₓ−Exₘᵢₙ₎]=(2.581−1.217)/(2.581−0.019)=(1.364)/(2.562)=0.53.

Alternatively, the same value may be calculated using the equation Mₛₐₘₚ=1−[(EXₛₐₘₚ−Exₘᵢₙ)/(EXₘₐₓ−EXₘᵢₙ)]. For example: 1−[(1.217−0.019)/(2.581−0.019)]=1−[(1.198)/(2.562)]=1−0.467=0.53. As this M is greater than the minimal M required for preventing transmission (0.495), this patient will not transmit the virus to the fetus.

TABLE 2
qRT PCR expression results of the 3 selected genes in
18 pregnant women infected with HCMV

| Women | Selected Genes | | |
|---|---|---|---|
| List | ISG15 | IFIT3 | USP18 |
| 16YM | 1.217 | 1.603 | 2.772 |
| 06RB | 0.106 | 0.214 | 0.370 |
| 08PA | 0.909 | 1.105 | 1.381 |
| 35PC | 0.283 | 0.342 | 1.241 |
| 42GM | 0.296 | 0.967 | 1.961 |
| 75TT | 0.065 | 0.039 | 0.147 |
| 17SA | 0.068 | 0.033 | 0.357 |
| 67MA | 0.019 | 0.054 | 0.342 |
| 68KA | 0.088 | 0.264 | 0.718 |
| 10GC | 0.597 | 1.213 | 1.353 |
| 13SR | 0.040 | 0.135 | 0.363 |
| 28RR | 0.025 | 0.069 | 0.136 |
| 32HL | 0.138 | 0.385 | 0.775 |
| 19TA | 1.442 | 0.997 | 2.895 |
| 43VE | 1.695 | 1.462 | 1.784 |
| 07GR | 2.119 | 2.442 | 1.158 |
| 37LH | 2.581 | 3.388 | 5.718 |
| 22SH | 1.224 | 1.359 | 8.311 |

### Example 4

Expression of IFN Signaling Genes in hCMV Infected Pregnant Women

A further analysis was conducted on total cellular RNA from stored frozen PMBC collected from 24 or 29 consent pregnant women (18 of which were described in the Example 3).

In order to obtain a clear distinction and a predictive tool, the inventors have analyzed in addition to the three genes described above, namely IFIT3, USP18 and ISG15, the expression level of additional four genes, EIF2AK2, HERC5, RSAD2 and MX1.

As shown herein below, analyses of the selected seven bio-markers expression by qRT-PCR and subsequent normalization strongly suggest that the hCMV infected pregnant women can be divided into two groups: women express low levels and women who express high levels of the bio-markers.

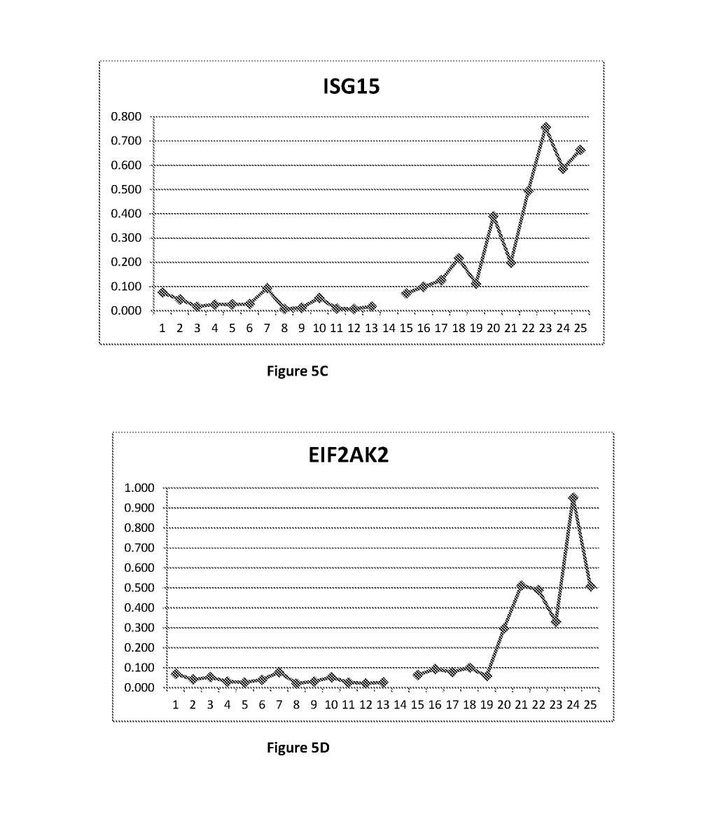

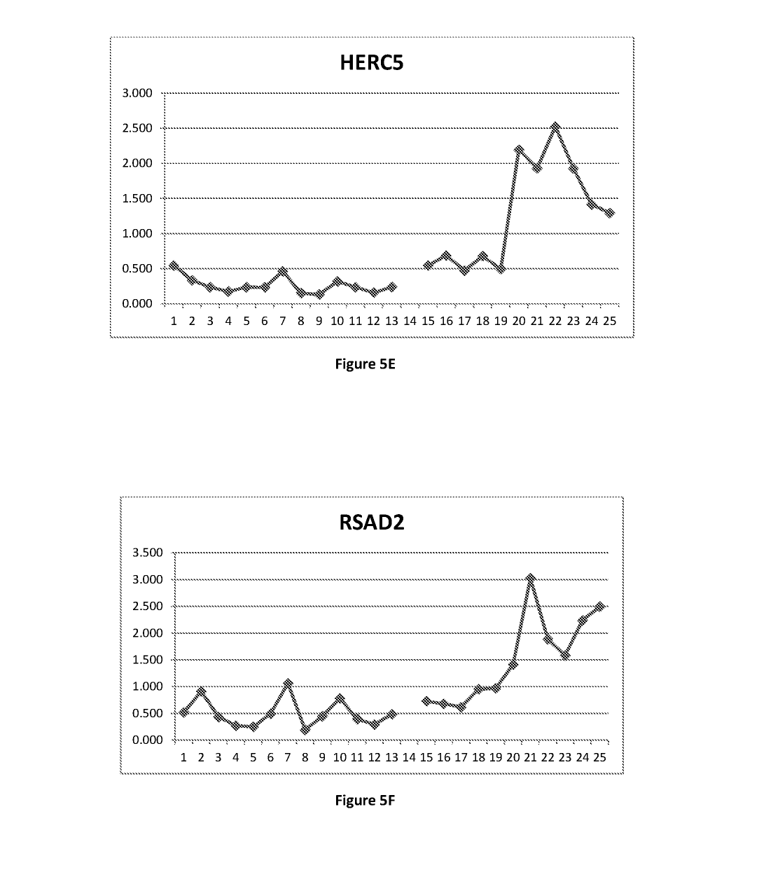

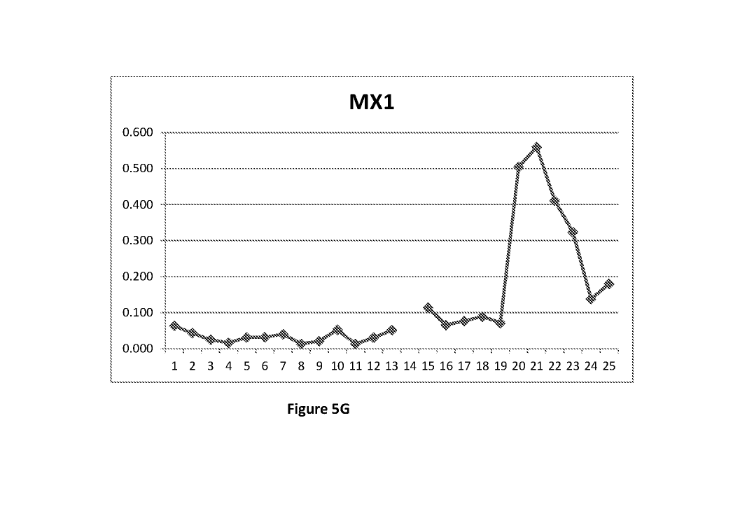

Specifically, the expression of each one of the seven genes, IFIT3, USP18, ISG15, EIF2AK2, HERC5, RSAD2 and MX1 as measured in 24 hCMV infected pregnant women is shown in FIGS. 5A to 5G, respectively. In these figures, pregnant women denoted as #1 to #13 were characterized as non-transmitters and pregnant women denoted as #15 to #25 were characterized as transmitters).

As shown in FIGS. 5A to 5G, the women population can be divided into two separate group: 11 transmitters with high expression level of the seven genes and 13 non transmitters with low expression level.

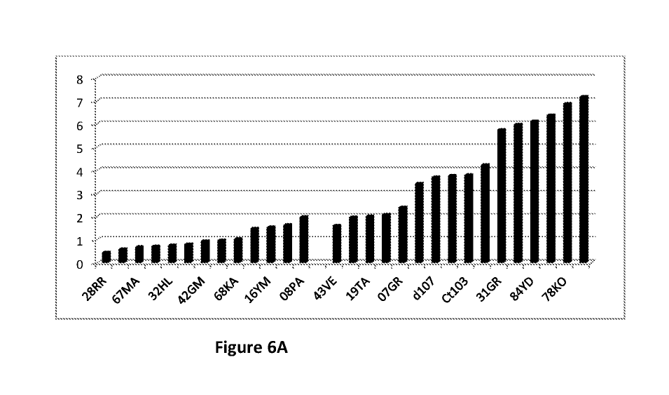

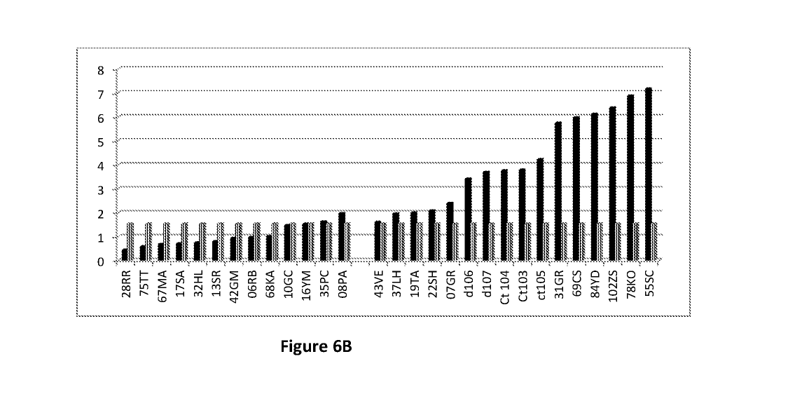

The inventors have further calculated the sum of all the seven genes. FIG. 6A shows the sum of the expression of seven selected gene (ISG15, IFIT3, USP18 EIF2AK2, HERC5, RSAD2 and MX1) in 29 hCMV infected pregnant women, emphasizing the differences between the two groups of women, with low value of the sum correlating with 13 non transmitters women and high value correlating with 16 transmitters women FIG. 6B shows the sum of the seven genes in each one of the women vs. a threshold bar (shown as dashed line) showing that a sum lower than the threshold is indicative of non-transmitters women whereas a sum higher than the threshold is indicative of transmitters women.

1. A method for the prediction and diagnosis of intrauterine-transmission of a viral pathogen in a mammalian subject, the method comprising:
(a) determining the level of expression of at least one of ISG15 ubiquitin-like modifier (ISG15), Interferon-induced protein with tetratricopeptide repeats 3 (IFIT3), Ubiquitin specific peptidase 18 (USP18), Eukaryotic Translation Initiation Factor 2 Alpha Kinase 2 (EIF2AK2), HECT And RLD Domain Containing E3 Ubiquitin Protein Ligase 5 (HERC5), Radical S-Adenosyl Methionine Domain Containing 2 (RSAD2) and MX Dynamin Like GTPase 1 (MX1) genes in a biological sample of said subject, to obtain an expression value Exₛₐₘₚ in said sample;
(b) calculating the M value of said sample (Mₛₐₘₚ), wherein said value indicates the ability of said subject to prevent intrauterine-transmission of said viral pathogen;
(c) providing a standard M (M_{stand}) value of non-transmitting subjects, said value indicates the minimal ability required for preventing intrauterine-transmission of said viral pathogen;
(d) determining if the M value of said sample (Mₛₐₘₚ) calculated in step (b) is any one of positive or negative with respect to the standard M (M_{stand}) value of non-transmitting subjects provided in (c);
wherein a positive value of Mₛₐₘₚ indicates that the subject is a non-transmitting subject and a negative value of Mₛₐₘₚ indicates that the subject is viral pathogen transmitting subject, thereby predicting intrauterine-transmission of said viral pathogen in said subject.

2. The method according to claim 1, wherein calculating the M value of said sample (Mₛₐₘₚ) is performed by the steps of:
(a) obtaining the expression value Exₛₐₘₚ of said sample ad determined in 1(a);
(b) providing a standard curve of expression values of said viral pathogen infected subjects;
(c) obtaining a maximal expression value Exₘₐₓ and a minimal expression value Exₘᵢₙ from said standard curve of (b); and
(d) calculating the Mₛₐₘₚ value of said sample, wherein Mₛₐₘₚ=1−[(Exₛₐₘₚ−Exₘᵢₙ)/(Exₘₐₓ−Exₘᵢₙ₎].

3. The method according to claim 1, wherein any one of (i) said viral pathogen is human cytomegalovirus (hCMV); (ii) said subject is a human female subject; (iii) said subject is a human female subject pregnant at early stage of gestation; (iv) wherein said sample is any one of blood cells and amniotic fluid.

4-5. (canceled)

6. The method according to claim 1,
wherein determining the level of expression of at least one of said ISG15, IFIT3, USP18, EIF2AK2, HERC5, RSAD2 and MX1 genes in a biological sample of said subject is performed by the step of contacting detecting molecules specific for said genes with a biological sample of said subject, or with any nucleic acid or protein product obtained therefrom,
wherein said detecting molecules are selected from isolated detecting nucleic acid molecules and isolated detecting amino acid molecules, optionally wherein said nucleic acid detecting molecule comprises isolated oligonucleotide/s, each oligonucleotide specifically hybridizes to a nucleic acid sequence of said at least one of ISG15, IFIT3, USP18, EIF2AK2, HERC5, RSAD2 and MX1 genes and optionally, to a control reference gene,
optionally wherein said detecting molecule is at least one of a pair of primers, at least one primer, nucleotide probes or any combinations thereof.

7-10. (canceled)

11. The method according to claim 1, wherein said first step (a) comprises determining the level of expression of at least one of ISG15, IFIT3 and USP18.

12. A kit comprising detecting molecules specific for determining the level of expression of ISG15, IFIT3 and USP18, genes in a biological sample.

13. The kit according to claim 12, further comprising detecting molecules specific for determining the level of expression of EIF2AK2, HERC5, RSAD2 and MX1.

14. The kit according to claim 12, further comprising at least one of:
(a) means for calculating the M value of a tested subject (Mₛₐₘₚ), wherein said value indicates the ability of said subject to prevent intrauterine-transmission of a viral pathogen;
(b) a standard M (M_{stand}) value of non-transmitting subjects, said value indicates the minimal ability required for preventing intrauterine-transmission of said viral pathogen;
(c) means for calculating a standard M_{stand} value for non-transmitting population;
(d) detecting molecules specific for determining the level of expression of at least one control reference gene in a biological sample; and
(e) at least one control sample.

15. The kit according to claim 14, wherein means for calculating the value of M of a tested subject (Mₛₐₘₚ) comprise at least one of:
(a) detecting molecules specific for determining the level of expression of ISG15, IFIT3 and USP18 genes, and optionally of EIF2AK2, HERC5, RSAD2 and MX1 genes in a biological sample for determining an expression value Exₛₐₘₚ in said sample;
(b) pre-determined calibration curve providing standard expression values of ISG15, IFIT3 and USP18 genes and optionally of EIF2AK2, HERC5, RSAD2 and MX1 genes in said viral pathogen infected subjects or predetermined maximal expression value Exₘₐₓ and a minimal expression value Exₘᵢₙ calculated from said standard curve; and
(c) a formula for calculating Mₛₐₘₚ value, wherein said formula is Mₛₐₘₚ=[(Exₛₐₘₚ−Exₘᵢₙ)/(Exₘₐₓ−Exₘᵢₙ₎].

16. The kit according to claim 12, wherein said viral pathogen is hCMV, optionally wherein said subject is an hCMV infected subject.

17. The kit according to claim 16, wherein said kit is a diagnostic kit for predicting intrauterine-transmission of hCMV in a female subject optionally pregnant at early stage of gestation.

18-20. (canceled)

21. The kit according to claim 12, further comprising any one of (i) instructions for use, wherein said instructions comprise at least one of:
(a) instructions for carrying out the detection and quantification of expression of said ISG15, IFIT3 and USP18 genes and optionally of said EIF2AK2, HERC5, RSAD2 and MX1 genes;
(b) instructions for carrying out the detection and quantification of expression of said at least one control reference gene;
(c) instructions for determining if the calculated M value of said sample (Mₛₐₘₚ) is any one of positive or negative with respect to the standard M (M_{stand}) value of non-transmitting subjects;
(ii) at least one reagent for conducting a nucleic acid amplification based assay selected from the group consisting of a Real-Time PCR, micro arrays, PCR, in situ Hybridization and Comparative Genomic Hybridization;
(iii) a solid support, wherein each of said detecting molecules is disposed in an array, optionally wherein said array of detecting molecules comprises a plurality of addressed vessels, optionally wherein said array of detecting molecules comprises a solid support holding detecting molecules in distinct regions.

22. The kit according to claim 12, wherein said detecting molecules comprise at least one of isolated detecting nucleic acid molecules and isolated detecting amino acid molecules, optionally wherein said detecting molecules are at least one of at least one primer, at least one pair of primers, at least one nucleotide probe and any combination thereof.

23. The kit according to claim 22, wherein said detecting molecules comprise isolated oligonucleotides, each said oligonucleotide specifically hybridize to a nucleic acid sequence of an RNA product of one of said ISG15, IFIT3 and USP18 genes and optionally of said EIF2AK2, HERC5, RSAD2 and MX1 genes.

24-28. (canceled)

29. The kit according to claim 12, wherein said sample is at least one of a blood sample and amniotic fluid.

30. The kit according to claim 29, wherein said sample is a blood sample, and wherein said kit comprises detecting molecule/s specific for determining the level of expression of ISG15, IFIT3 and USP18 genes and optionally of EIF2AK2, HERC5, RSAD2 and MX1 genes in said blood sample.

31. An array of detecting molecules specific for ISG15, IFIT3 and USP18, genes, wherein said detecting molecules are isolated detecting nucleic acid molecules or isolated detecting amino acid molecule/s.

32. The array according to claim 31, further comprising detecting molecules specific for EIF2AK2, HERC5, RSAD2 and MX1 genes.

33. The array according to claim 31, comprising any one of (i) a plurality of addressed vessels containing said detecting molecule/s (ii) a solid support holding detecting molecules in distinct regions.

34-36. (canceled)

37. The array according claim 31, for the prediction and diagnosis of intrauterine-transmission of human cytomegalovirus (hCMV) in a mammalian subject.
